(19) United States
(12) Patent Application Publication
(10) Pub. No.: US 2004/0253245 A1
(43) Pub. Date: Dec. 16, 2004

(54) Modulators

(76) Inventors: Emmanuel Cyrille Pascal Briend, Cambridge (GB); Brian Robert Champion, Cambridge (GB); Roberto Celeste Ercole Solari, Cambridge (GB)

Correspondence Address: FROMMER LAWRENCE & HAUG, 745 FIFTH AVENUE- 10TH FL., NEW YORK, NY, 10151, US

(21) Appl. No.: 10/812,144

(22) Filed: Mar. 29, 2004

Related U.S. Application Data
(63) Continuation-in-part of application No. PCT/GB02/04390, filed on Sep. 27, 2002.

(30) Foreign Application Priority Data
Sep. 28, 2001 (GB) 0123379.0

(51) Int. Cl. A61K 39/395; A61K 48/00; A61K 38/17; A61K 38/12

(52) U.S. Cl. 424/146.1; 514/12; 514/44; 514/9

(57) ABSTRACT

Provided are methods of using a modulator of Notch IC protease activity in the manufacture of a medicament for use in immunotherapy and methods of detecting such a modulator.

### CROSS REFERENCE TO RELATED APPLICATIONS

This application is a continuation-in-part of International Application No. PCT/GB02/04390, filed on Sep. 27, 2002, published as WO 03/029293 on Apr. 10, 2003, and claiming priority to GB application Serial No. 0123379.0, filed on Sep. 28, 2001. Reference is made to U.S. application Ser. No. 09/310,685, filed on May 4, 1999, Ser. No. 09/870,902, filed on May 31, 2001, Ser. No. 10/013,310, filed on Dec. 7, 2001, Ser. No. 10/147,354, filed on May 16, 2002, Ser. No. 10/357,321, filed on Feb. 3, 2002, Ser. No. 10/682,230, filed on Oct. 9, 2003, Ser. No. 10/720,896, filed on Nov. 24, 2003, and Ser. No. 10/763,362, 10/764,415 and 10/765,727, all filed on Jan. 23, 2004.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer 's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to the use of modulators of Notch intracellular domain (Notch IC) protease activity including modulators of presenilin and presenilin-dependent gamma secretase activity.

### BACKGROUND OF THE INVENTION

The etiological basis of Alzheimer's disease (AD) is not yet clear, however, a major portion of AD can be attributed to genetic factors. Familial Alzheimer's disease (FAD) is genetically heterogeneous and can be categorised according to age-of-onset using 60 years as a cut-off. The early-onset FAD genes include the amyloid β-protein precursor (APP) gene on chromosome 21, the presenilin 1 (PS1) gene on chromosome 14, and the presenilin 2 (PS2) gene on chromosome 1. Approximately 50% of early-onset FAD is accounted for by defects in these genes, with the majority occurring in the PS1 gene.

Presenilins have been implicated in Notch signalling and more particularly in the proteolysis of Notch to release its intracellular domain to the nucleus. Moreover gamma-secretases have been reported as also affecting this step in the Notch signalling pathway (DeStrooper). This is reviewed in Selkoe. Notch signal transduction also plays a critical role in cell fate determination in vertebrate and invertebrate tissues. Notch is expressed at many stages of Drosophila embryonic and larval development and in many different cells implying a wide range of functions including an important role in neurogenesis and in the differentiation of mesodermal and endodermal cells. Recent investigations have therefore concentrated on antagonists of presenilin in order to treat Alzheimer's diseases and other neural-associated diseases, and for altering the fate of a cell (see for example WO01/03743 and Hadland et al.).

During maturation in the thymus, T cells acquire the ability to distinguish self-antigens from those that are non-self, a process termed “self tolerance”. Tolerance to a non-self antigen, however, may be induced by immunisation under specific conditions with a peptide fragment comprising that antigen. In autoimmune diseases such as multiple sclerosis, rheumatoid arthritis or diabetes, there is a failure of the proper regulation of tolerance. Improved treatment methods for re-establishing tolerance are desirable for autoimmune diseases. Similarly in allergic conditions and for transplantation of an organ or tissue from a donor individual, induction of tolerance to particular foreign antigens or profiles of foreign antigens is desirable.

The expression on the cell surface of normal adult cells of the peripheral immune system of Notch and its ligands, Delta and Serrate, suggests a role for these proteins in T cell acquired immunocompetence (Hoyne et al. (2000) Int. Immunol, 12:177-185). T cells express Notch mRNA constitutively. These observations reinforce the view that the Notch receptor ligand family continues to regulate cell fate decisions in the immune system beyond embryonic development (Ellisen) with Notch signalling playing a central role in the induction of peripheral unresponsiveness (tolerance or anergy), linked suppression and infectious tolerance.

Linked suppression occurs when an intact antigenic molecule is used for challenge immunisation and is characterised by cells being tolerised against, not only the target antigen, but also other, non-target regions of the antigenic molecule (Hoyne et al. (2000)). Infectious tolerance is a process whereby it is possible to generate a class of regulatory T cells which are able to transmit antigen-specific tolerance to other neighbouring T cells (Qin and WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins. Delta or Serrate induced T cells specific to one antigenic epitope are also able to transfer tolerance to T cells recognising other epitopes on the same or related antigens, a phenomenon termed “epitope spreading”.

Thus, as described in WO 98/20142, WO 00/36089 and WO 01/35990, manipulation of the Notch signalling pathway can be used in immunotherapy and in the prevention and/or treatment of T cell mediated diseases. In particular, allergy, autoimmunity, graft rejection, tumour induced aberrations to the T cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted. Notch ligand expression also plays a role in cancer. Indeed, upregulated Notch ligand expression has been observed in some tumour cells. These tumour cells are capable of rendering T cells unresponsive to restimulation with a specific antigen, thus providing a possible explanation of how tumour cells prevent normal T cell responses. Downregulation of Notch signalling in vivo in T cells may be used to prevent tumour cells from inducing immunotolerance in those T cells that recognise tumour-specific antigens. In turn, this allows the T cells to mount an immune response against the tumour cells (WO00/35990). A description of the Notch signalling pathway and conditions affected by it may be found in our published PCT Applications WO 98/20142, WO 00/36089 and WO 0135990. The text of each of PCT/GB97/03058 (WO 98/20142), PCT/GB99/04233 (WO 00/36089) and PCT/GB00/04391 (WO 0135990) is hereby incorporated herein by reference.

The present invention seeks to provide further methods of modulating the immune system by modification of the Notch signalling pathway.

### SUMMARY OF THE INVENTION

According to one aspect of the present invention, there is provided the use of a modulator of Notch IC protease activity for the manufacture of a medicament for use in immunotherapy.

Put another way the present invention provides a method of immunotherapy comprising administering to an individual in need of the same an effective amount of a modulator of Notch IC protease activity.

In a preferred embodiment the invention relates to use of a modulator of presenilin activity.

In a further embodiment the invention relates to use of a modulator of presenilin-dependent gamma secretase activity.

By “immunotherapy” we include the diagnosis, prevention or treatment of diseases, infections or conditions affected by the immune system.

In one embodiment, immunotherapy will involve the control of T cell activity including the treatment of a T cell mediated disease or infection, such as a T cell mediated disease or infection caused by any one or more of allergy, autoimmunity, graft rejection, tumour induced aberrations to the T cell and infectious diseases.

The term “Notch IC protease” as used herein means an enzyme or enzyme complex which acts proteolytically to cleave the Notch receptor to cause the release of all or part of the intracellular (IC) domain from the Notch receptor so as to activate the Notch signalling pathway. Enzymes which are understood to participate in such cleavage include the presenilins and gamma-secretase enzymes, and presenilin-dependent gamma-secretase enzymes or complexes.

The term “presenilin-dependent gamma-secretase” as used herein means an enzyme having gamma secretase proteolytic activity which requires presenilin for activity or activation. The presenilin may for example be required as a co-activator or as part of an enzyme complex.

In one embodiment, the modulator of Notch IC protease activity is used in combination with a modulator of the Notch signalling pathway. For example, an inhibitor of Notch IC protease activity may be used in combination with an activator of the Notch receptor to increase relative signalling along the CSL-independent pathway (for example to increase Notch-induced signalling along the Ras-MAP Kinase pathway).

Recent studies have shown that Notch activation generally requires that the six cdc10/ankyrin repeats of the Notch intracellular domain reach the nucleus and participate in transcriptional activation. An important site of proteolytic cleavage on the intracellular tail of Notch 1 has been identified between gly1743 and val1744 (termed site 3, or S3) (Schroeter). It is believed that the proteolytic cleavage step that releases the NotchIC for nuclear entry preferably involves Presenilin activity.

The Notch intracellular domain has been shown to accumulate in the nucleus where it forms a transcriptional regulator complex with other transcription factors such as the CSL family member CBF1 (suppressor of hairless, Su(H) in Drosophila, Lag-2 in C. elegans) (Schroeter; Struhl) and Mastermind (MAML1/2). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

Preferably the modulator of Notch IC protease activity modulates Notch IC protease activity in immune cells, preferably lymphocytes, and more preferably peripheral T-cells.

In one embodiment, the modulator of Notch IC protease may, for example, be an agent which binds at or near to the active site of the enzyme to reduce activity. Such an agent may be a so-called “small molecule” preferably having a molecular weight of less than about 1000 Da and preferably of less than about 500 Da. Suitably such an inhibitor may, for example, have an IC₅₀ of less than about 500 micromolar, suitably less than about 100 micromolar, preferably less than about 10 micromolar.

Examples of presenilin proteins which may be modulated in the present invention include Presenilin-1 (PS1) and Presenilin-2 (PS2).

The modulator of Notch IC protease activity will preferably be selected from polypeptides and fragments thereof, linear peptides, cyclic peptides, and nucleic acids which encode therefor, synthetic and natural compounds including low molecular weight organic or inorganic compounds and antibodies. The modulator may for example be an agonist or an antagonist of presenilin or presenilin-dependent gamma-secretase, optionally in combination with an agent capable of respectively up-regulating or down-regulating the Notch signalling pathway respectively.

Examples of agonists of presenilin which may be used in the present invention include Nicastrin or ALG-3 or a nucleic acid sequence which encodes therefor. An example of an antagonist of presenilin which may be used in the present invention is 26S proteasome or a nucleic acid sequence which encodes therefor. Synthetic inhibitors include, for example, the difluoro ketone inhibitor described in Citron et al., and Wolfe et al. having the formula:

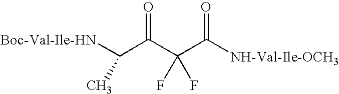

Other synthetic inhibitors include those described in Sinha and Liederburg (2-Naphthoyl-VF-CHO, N-(2-Naphthoyl)-Val-phenylalaninal and N-Benzyloxycarbonyl-Leu-phenylalaninal Z-LF-CHO); the inhibitors described in Esler et al.; the inhibitors described in Figueiredo-Pereira et al., (N-Benzyloxycarbonyl-Leu-leucinal Z-LL-CHO); the inhibitors described in Higaki et al., (N-trans-3,5-Dimethoxycinnamoyl)-Ile-leucinal t-3,5-DMC-IL-CHO); the inhibitors described in Murphy et al., (Boc-GVV-CHO N-tert-Butyloxycarbonyl-Gly-Val-Valinal); and the inhibitors described in Riston et al., (1-(S)-endo-N-(1,3,3)-Trimethylbicyclo[2.2.1]hept-2-yl)-4-fluorophenyl Sulfonamide).

Secretase activity may be inhibited by metalloproteases.

Agents capable of up-regulating expression of the Notch signalling pathway and which may be used in the present invention include, but are not limited to, Notch ligands of the Serrate/Jagged and Delta families, Noggin, Chordin, Follistatin, Xnr3, FGF and derivatives, fragments, variants and homologues thereof, and immunosuppressive cytokines, or a combination thereof, and nucleic acid sequences which encode therefor. Agents capable of down-regulating the Notch signalling pathway and which may be used in the present invention include, but are not limited to, a Toll-like receptor, a cytokine, a bone morphogenetic protein (BMP), a BMP receptor or an activin or a nucleic acid sequence which encodes therefor.

In another aspect of the present invention there is provided a modulator of Notch IC protease activity for use in immunotherapy.

In another aspect of the present invention there is provided a modulator of Notch IC protease activity for use in affecting a cell mediated disease, condition or infection.

In another aspect of the present invention there is provided a modulator of Notch IC protease activity for use in affecting linked suppression.

In another aspect of the present invention, there is provided a modulator of Notch IC protease activity for use in affecting infectious tolerance.

In one embodiment, the modulator of presenilin or presenilin-dependent gamma-secretase may be used in combination with a modulator of the Notch signalling pathway as described above.

In another aspect of the present invention, there is provided a method for producing a lymphocyte or antigen presenting cell (APC) having the ability to induce tolerance to an allergen, antigen or antigenic determinant which method comprises incubating a lymphocyte or APC obtained from a human or animal patient with (i) an agonist of presenilin or presenilin-dependent gamma-secretase and optionally an agent capable of up-regulating endogenous Notch or Notch ligand in the lymphocyte and/or APC and (ii) the allergen, antigen or antigenic determinant.

In one embodiment, the APC produced by the above method will be capable of inducing T cell tolerance. As such, there is further provided a method for producing ex vivo a T cell having tolerance to an allergen, antigen or antigenic determinant, which method comprises incubating a T cell obtained from a human or animal patient with an antigen presenting cell (APC) in the presence of (i) an agonist of presenilin or presenilin-dependent gamma-secretase and optionally an agent capable of up-regulating expression of an endogenous Notch or Notch ligand in the APC and/or T cell and (ii) the allergen or antigen.

In another aspect of the present invention, there is provided a method for producing a lymphocyte or APC having tolerance to an allergen, antigen or antigenic determinant which method comprises incubating a lymphocyte or APC obtained from a human or animal patient with a lymphocyte or APC produced by any one of the above described methods.

In one embodiment, there is provided a method as described above for producing ex vivo a T cell having the ability to induce tolerance to an allergen or antigen which method comprises incubating a T cell obtained from a human or animal patient with a T cell produced by the method of any one of the above described methods.

In a further aspect of the present invention, there is provided the use of a lymphocyte or APC produced by any one of the methods of the invention in suppressing an immune response in a mammal to the allergen or antigen.

In another aspect of the present invention, there is provided a method of treating a patient suffering from a disease characterised by inappropriate lymphocyte activity which method comprises administering to the patient a lymphocyte produced by the method of the invention.

In another aspect of the invention, there is provided a method for enhancing the reactivity of a T cell toward a tumour cell, which method comprises:

(a) isolating a T cell from a patient having said tumour cell present in their body;

(b) exposing the T cell to a modulator of Notch IC protease activity, optionally in the presence of an agent which is capable of reducing or preventing expression or interaction of an endogenous Notch or Notch ligand in the T cell; and

(c) re-introducing the T cell into the patient;

wherein the T cell comprises a T cell receptor specific for a tumour antigen expressed by the tumour cell.

In another aspect of the present invention, there is provided a method for enhancing the reactivity of a T cell toward a tumour cell, which method comprises:

(a) isolating an antigen presenting cell (APC) from a tumour present in the body of a patient;

(b) exposing the APC to a modulator of Notch IC protease activity, optionally in the presence of an agent which is capable of reducing or preventing expression or interaction of an endogenous Notch or Notch ligand in the T cell when the T cell is contacted with the APC; and

(c) re-introducing the APC into the patient.

In another aspect of the present invention, there is provided a method for enhancing the reactivity of a T cell toward a tumour cell, which method comprises:

(a) isolating a tumour cell from a tumour present in the body of a patient;

(b) exposing the tumour cell to a modulator of Notch IC protease activity, optionally in the presence of an agent which is capable of reducing or preventing expression or interaction of an endogenous Notch or Notch ligand in the T cell when the T cell is contacted with the tumour cell; and

(c) re-introducing the tumour cell into the patient.

In one embodiment, the T cell used in the above methods is a tumour infiltrating lymphocyte (TIL).

In another aspect of the present invention, there is provided a method of vaccinating a patient against a tumour which method comprises:

(a) administering a tumour antigen or antigenic determinant expressed by the tumour to a patient (suitably to the skin); and

(b) exposing the APC present in the patient to a modulator of Notch IC protease activity.

In another aspect of the present invention, there is provided an assay method for modulators of Notch IC protease activity comprising contacting a Notch IC protease, in the presence of Notch and optionally a modulator of the Notch signalling pathway, with a candidate compound and determining if the compound affects the Notch signalling pathway. Suitably the assay is conducted in an immune cell, for example a lymphocyte or lymphocytic cell line.

In yet another aspect of the present invention, there is provided an assay method for identifying substances that affect the interaction of a presenilin-interacting protein or presenilin-dependent gamma-secretase-interacting protein with a presenilin protein or presenilin-dependent gamma-secretase, respectively, comprising:

(a) providing a preparation containing: a presenilin protein or presenilin-dependent gamma-secretase; a presenilin-interacting protein or presenilin-dependent gamma-secretase-interacting protein, respectively; and a candidate substance; and

(b) detecting whether said candidate substance affects said interaction of said presenilin-interacting protein or presenilin-dependent gamma-secretase-interacting protein with said presenilin protein or presenilin-dependent gamma-secretase.

In one embodiment, the presenilin-interacting protein is Notch or a member of the Notch signalling pathway.

In a further aspect of the present invention, there is provided the use of a Notch IC protease modulator such as a presenilin or presenilin-dependent gamma-secretase modulator identifiable using any one of the above assay methods in any one of the uses or methods of the invention.

In a yet further aspect of the present invention, there is provided a kit comprising, in one or more containers, (a) a modulator of the Notch signalling pathway and (b) a modulator of Notch IC protease activity.

According to a further aspect of the invention, there is provided the use of a modulator of Notch IC protease activity for the manufacture of a medicament for modulation of an immune response. In one embodiment, there is provided the use of an inhibitor of Notch IC protease activity for the manufacture of a medicament for enhancement of an immune response. Alternatively, there is provided the use of an enhancer of Notch IC protease activity for the manufacture of a medicament for reduction of an immune response.

According to a further aspect of the invention, there is provided the use of a modulator of Notch IC protease activity for the manufacture of a medicament for modulation of an immune response to a selected antigen or antigenic determinant.

According to a further aspect of the invention, there is provided the use of a modulator of Notch IC protease activity for the manufacture of a medicament for modulation of lymphocyte activity.

According to a further aspect of the invention, there is provided the use of a modulator of Notch IC protease activity for the manufacture of a medicament for modulation of T-cell activity such as effector T-cell activity.

Thus, in one embodiment, there is provided the use of a modulator of Notch IC protease activity for the manufacture of a medicament for modulation of helper (Th) T-cell activity. In one embodiment, this may comprise the use of an inhibitor of Notch IC protease activity for the manufacture of a medicament for increase of helper (Th) T-cell activity. In an alternative embodiment, this may comprise the use of an enhancer of Notch IC protease activity for the manufacture of a medicament for reducing helper (Th) T-cell activity.

According to a further aspect of the invention, there is provided the use of a modulator of Notch IC protease activity for the manufacture of a medicament for modulation of cytotoxic (Tc) T-cell activity. In one embodiment, this may comprise the use of an inhibitor of Notch IC protease activity for the manufacture of a medicament for increasing cytotoxic (Tc) T-cell activity. In an alternative embodiment, this may comprise the use of an enhancer of Notch IC protease activity for the manufacture of a medicament for decreasing cytotoxic (Tc) T-cell activity.

According to a further aspect of the invention, there is provided the use of a modulator of Notch IC protease activity for the manufacture of a medicament for modulation of regulatory (T reg) T-cell activity. In one embodiment, this may comprise the use of an inhibitor of Notch IC protease activity for the manufacture of a medicament for reduction of regulatory (T reg) T-cell activity. In an alternative embodiment, this may comprise the use of an enhancer of Notch IC protease activity for the manufacture of a medicament for increasing regulatory (T reg) T-cell activity.

According to a further aspect of the invention, there is provided the use of a modulator of Notch IC protease activity for the manufacture of a medicament for modulation of Tr1 or Th3 regulatory T-cell activity. In one embodiment, this may comprise the use of an inhibitor of Notch IC protease activity for the manufacture of a medicament for inhibition of Tr1 or Th3 regulatory T-cell activity. In an alternative embodiment, this may comprise the use of an enhancer of Notch IC protease activity for the manufacture of a medicament for enhancing Tr1 or Th3 regulatory T-cell activity.

According to a further aspect of the invention, there is provided the use of a modulator of Notch IC protease activity for the manufacture of a medicament for modulation of cytokine expression, such as lymphokine expression or monokine expression. In particular, there is provided the use of a modulator of Notch IC protease activity for the manufacture of a medicament for modulation of Notch-mediated cytokine expression.

The term “Notch mediated” as used herein means effects caused or influenced primarily or substantially by activation or inhibition of the Notch signalling pathway, and preferably by the degree of activation of the Notch receptor.

According to one such aspect of the invention, there is provided the use of a modulator of Notch IC protease activity for the manufacture of a medicament for modulation of expression of a cytokine selected from IL-10, IL-5, IL-4, IL-2, TNF-alpha, IFN-gamma or IL-13. Preferably there is provided the use of a modulator of Notch IC protease activity for the manufacture of a medicament for modulation of Notch-mediated expression of a cytokine selected from IL-10, IL-5, IL-4, IL-2, TNF-alpha, IFN-gamma or IL-13.

Thus, in one aspect, the invention provides the use of an inhibitor of Notch IC protease activity for the manufacture of a medicament for decrease of IL-10 or IL-4 expression, preferably for decrease of Notch-mediated IL-10 or IL-4 expression. Alternatively there is provided the use of an activator of Notch IC protease activity for the manufacture of a medicament for increase of IL-10 or IL-4 expression, preferably for increase of Notch-mediated IL-10 or IL-4 expression.

According to a further aspect of the invention, there is provided the use of an inhibitor of Notch IC protease activity for the manufacture of a medicament for increase of expression of a cytokine selected from IL-2, IL-5, TNF-alpha, IFN-gamma or IL-13, prferably for increase of Notch-mediated expression of a cytokine selected from IL-2, IL-5, TNF-alpha, IFN-gamma or IL-13. Alternatively, there is provided the use of an activator of Notch IC protease activity for the manufacture of a medicament for decrease of expression of a cytokine selected from IL-2, IL-5, TNF-alpha, IFN-gamma or IL-13, preferably for decrease of Notch-mediated expression of a cytokine selected from IL-2, IL-5, TNF-alpha, IFN-gamma or IL-13.

According to a further aspect of the invention, there is provided the use of an enhancer of Notch IC protease activity for the manufacture of a medicament for generating an immune modulatory cytokine profile with increased IL-10 expression and reduced IL-5 expression. According to a further aspect of the invention, there is provided the use of an enhancer of Notch IC protease activity for the manufacture of a medicament for generating an immune modulatory cytokine profile with increased IL-10 expression and reduced IL-2, IFN-gamma, IL-5, IL-13 and TNF-alpha expression.

Alternatively, there is provided the use of an inhibitor of Notch IC protease activity for the manufacture of a medicament for generating an immune modulatory cytokine profile with decreased IL-10 expression and increased IL-5 expression.

The invention further provides the use of an inhibitor of Notch IC protease activity for the manufacture of a medicament for generating an immune modulatory cytokine profile with decreased IL-10 expression and increased IL-2, IFN-gamma, IL-5, IL-13 and TNF-alpha expression.

According to a further aspect of the invention, there is provided the use of an enhancer of Notch IC protease activity in the manufacture of a medicament for treatment of inflammation or an inflammatory condition.

According to a further aspect of the invention, there is provided the use of a combination of:

i) an enhancer or inhibitor of Notch IC protease activity; and

ii) an antigen or antigenic determinant or a polynucleotide coding for an antigen or antigenic determinant;

in the manufacture of a medicament for modulation of the immune response to the antigen or antigenic determinant.

According to a further aspect of the invention, there is provided the use of an enhancer or inhibitor of Notch IC protease activity in the manufacture of a medicament for modulation of the immune system in simultaneous, contemporaneous, separate or sequential combination with an antigen or antigenic determinant or a polynucleotide coding for an antigen or antigenic determinant.

Suitably, the modulator of Notch IC protease activity may be administered to a patient in vivo. Alternatively the modulator of Notch IC protease activity may be administered to a cell ex vivo.

According to a further aspect of the invention, there is provided a method of immunotherapy comprising administering a modulator of Notch IC protease activity. Suitably, the method comprises administering a modulator of Notch IC protease activity in combination with a modulator of the Notch signalling pathway. Suitably, the modulator may be an agonist of presenilin or presenilin-dependent gamma-secretase, optionally in combination with an agent capable of up-regulating the Notch signalling pathway. The agonist of presenilin may, for example, be a polypeptide selected from Nicastrin or ALG-3 or a nucleic acid sequence that encodes therefor.

Alternatively, the modulator may be an antagonist of presenilin or presenilin-dependent gamma-secretase, optionally in combination with an agent capable of down-regulating the Notch signalling pathway. The antagonist of presenilin may for example be 26S proteasome or a component thereof or Sel 10 or a nucleic acid sequence which encodes therefor.

Alternatively the antagonist may be an agent which binds to a Notch IC protease, for example at or near to the active site, so as to reduce activity.

According to a further aspect of the invention, there is provided a method for modulating an immune response by administering a modulator of Notch IC protease activity. In one such embodiment, the invention provides a method for modulating an immune response to a selected antigen or antigenic determinant by administering a modulator of Notch IC protease activity.

According to a further aspect of the invention, there is provided a method for modulating lymphocyte, and preferably T-cell, activity by administering a modulator of Notch IC protease activity.

Thus, in one embodiment, the invention provides a method for modulating effector T-cell activity by administering a modulator of Notch IC protease activity.

In a further embodiment, the invention provides a method for modulating helper (Th) T-cell activity by administering a modulator of Notch IC protease activity. In one such aspect, there is provided a method for increasing helper (Th) T-cell activity by administering an inhibitor of Notch IC protease activity. Alternatively there is provided a method for decreasing helper (Th) T-cell activity by administering an enhancer of Notch IC protease activity.

The invention further provides a method for modulating cytotoxic (Tc) T-cell activity by administering a modulator of Notch IC protease activity. In one such aspect there is provided a method for increasing cytotoxic (Tc) T-cell activity by administering an inhibitor of Notch IC protease activity. Alternatively, there is provided a method for decreasing cytotoxic (Tc) T-cell activity by administering an enhancer of Notch IC protease activity.

According to a further aspect of the invention, there is provided a method for modulating regulatory (T reg) T-cell activity by administering a modulator of Notch IC protease activity.

In one such embodiment, there is provided a method for decreasing regulatory (T reg) T-cell activity by administering an inhibitor of Notch IC protease activity. Alternatively, there is provided a method for increasing regulatory (T reg) T-cell activity by administering an enhancer of Notch IC protease activity. The regulatory T-cells may for example be Tr1 or Th3 T cells

According to a further aspect of the invention, there is provided a method for modulating cytokine expression, such as lymphokine or monokine expression, by administering a modulator of Notch IC protease activity. In particular there is provided a method for modulating Notch-mediated cytokine expression by administering a modulator of Notch IC protease activity.

The invention further provides a method for modulating expression of a cytokine selected from IL-10, IL-5, IL-4, IL-2, TNF-alpha, IFN-gamma or IL-13 by administering a modulator of Notch IC protease activity. Preferably the cytokine expression which is modulated is Notch-mediated cytokine expression.

Thus, in a further aspect there is provided a method for decreasing IL-10 or IL-4 expression, preferably for decreasing Notch-mediated IL-10 or IL-4 expression, by administering an inhibitor of Notch IC protease activity. In one such embodiment, the invention provides a method for increasing IL-10 or IL-4 expression by administering an activator of Notch IC protease activity. Alternatively, there is provided a method for increasing Notch-mediated IL-10 or IL-4 expression by administering an activator of Notch IC protease activity.

According to a further aspect of the invention, there is provided a method for increasing expression of a cytokine selected from IL-2, IL-5, TNF-alpha, IFN-gamma or IL-13 by administering an inhibitor of Notch IC protease activity. Preferably the cytokine expression which is modulated is Notch-mediated expression.

Alternatively, there is provided a method for decreasing expression of a cytokine selected from IL-2, IL-5, TNF-alpha, IFN-gamma or IL-13 by administering an activator of Notch IC protease activity.

According to a further aspect of the invention, there is provided a method for generating an immune modulatory cytokine profile with increased IL-10 expression and reduced IL-5 expression by administering an enhancer of Notch IC protease activity.

According to a further aspect of the invention, there is provided a method for generating an immune modulatory cytokine profile with increased IL-10 expression and reduced IL-2, IFN-gamma, IL-5, IL-13 and TNF-alpha expression by administering an enhancer of Notch IC protease activity.

According to a further aspect of the invention, there is provided a method for generating an immune modulatory cytokine profile with decreased IL-10 expression and increased IL-5 expression by administering an inhibitor of Notch IC protease activity.

According to a further aspect of the invention, there is provided a method for generating an immune modulatory cytokine profile with decreased IL-10 expression and increased IL-2, IFN-gamma, IL-5, IL-13 and TNF-alpha expression by administering an inhibitor of Notch IC protease activity.

According to a further aspect of the invention, there is provided a method for treating inflammation or an inflammatory condition by administering an enhancer of Notch IC protease activity.

According to a further aspect of the invention, there is provided a method for modulating the immune response to an antigen or antigenic determinant by simultaneously, contemporaneously, separately or sequentially administering a combination of:

i) a modulator of Notch IC protease activity; and

ii) an antigen or antigenic determinant or a polynucleotide coding for an antigen or antigenic determinant.

According to a further aspect of the invention there is provided a method for enhancing the reactivity of a T cell toward a tumour cell which method comprises:

(a) isolating a T cell from a patient having said tumour cell present in their body;

(b) exposing the T cell to a modulator of Notch IC protease activity, optionally in the presence of an agent which is capable of reducing or preventing expression or interaction of an endogenous Notch or Notch ligand in the T cell; and

(c) re-introducing the T cell into the patient;

wherein the T cell comprises a T cell receptor specific for a tumour antigen expressed by the tumour cell.

According to a further aspect of the invention there is provided a method for enhancing the reactivity of a T cell toward a tumour cell which method comprises:

(a) isolating an antigen presenting cell (APC) from a tumour present in the body of a patient;

(b) exposing the APC to a modulator of Notch IC protease activity, optionally in the presence of an agent which is capable of reducing or preventing expression or interaction of an endogenous Notch or Notch ligand in the T cell when the T cell is contacted with the APC; and

(c) re-introducing the APC into the patient.

According to a further aspect of the invention there is provided a method for enhancing the reactivity of a T cell toward a tumour cell which method comprises:

(a) isolating a tumour cell from a tumour present in the body of a patient;

(b) exposing the tumour cell to a modulator of Notch IC protease activity, optionally in the presence of an agent which is capable of reducing or preventing expression or interaction of an endogenous Notch or Notch ligand in the T cell when the T cell is contacted with the tumour cell; and

(c) re-introducing the tumour cell into the patient.

According to a further aspect of the invention there is provided a method of vaccinating a patient against a tumour which method comprises:

(a) administering a tumour antigen expressed by the tumour to a patient; and

(b) exposing the APC present in the patient to a modulator of Notch IC protease activity, optionally in the presence of an agent which is capable of reducing or preventing expression, interaction or processing of Notch or a Notch ligand in a T cell.

According to a further aspect of the invention there is provided a method for assaying modulators of Notch IC protease activity comprising contacting a presenilin or presenilin-dependent gamma-secretase, respectively, in the presence of Notch and a modulator of the Notch signalling pathway, with a candidate compound and determining if the compound affects the Notch signalling pathway.

According to a further aspect of the invention there is provided a method for identifying substances that affect the interaction of a presenilin interacting protein or presenilin-dependent gamma-secretase interacting protein with a presenilin protein or presenilin-dependent gamma-secretase, respectively, comprising:

(a) providing a preparation containing: a presenilin protein or presenilin-dependent gamma-secretase; a presenilin-interacting protein or presenilin-dependent gamma-secretase, respectively; and a candidate substance; and

(b) detecting whether said candidate substance affects said interaction of said presenilin-interacting protein or presenilin-dependent gamma-secretase-interacting protein with said presenilin protein or presenilin-dependent gamma-secretase.

Suitably, the presenilin-interacting protein is Notch or a member of the Notch signalling pathway.

Preferably, the assay is conducted using an immune cell.

According to a further aspect of the invention, there is provided the use of a presenilin or presenilin-dependent gamma-secretase modulator identifiable using such an assay method in any of the uses or methods as described above.

According to a further aspect of the invention, there is provided a kit comprising in one or more containers (a) a modulator of the Notch signalling pathway and (b) a modulator of Notch IC protease activity.

According to a further aspect of the invention, there is provided a product comprising:

i) a modulator of Notch IC protease activity; and

ii) an antigen or antigenic determinant or a polynucleotide coding for an antigen or antigenic determinant;

as a combined preparation for simultaneous, contemporaneous, separate or sequential use for modulation of the immune response to said antigen or antigenic determinant.

According to a further aspect of the invention, there is provided a pharmaceutical composition comprising:

i) a modulator of Notch IC protease activity; and

ii) an antigen or antigenic determinant or a polynucleotide coding for an antigen or antigenic determinant;

as a combined preparation for simultaneous, contemporaneous, separate or sequential use for modulation of the immune response to said antigen or antigenic determinant.

According to a further aspect of the invention, there is provided a pharmaceutical composition comprising:

i) a modulator of Notch IC protease activity;

ii) an antigen or antigenic determinant or a polynucleotide coding for an antigen or antigenic determinant; and

iii) a pharmaceutically acceptable carrier.

According to a further aspect of the invention, there is provided a pharmaceutical kit comprising a modulator of Notch IC protease activity and an antigen or antigenic determinant or a polynucleotide coding for an antigen or antigenic determinant.

According to a further aspect of the invention, there is provided the use of a modulator of Notch IC protease activity in the manufacture of a medicament for use as an immunostimulant.

According to a further aspect of the invention, there is provided the use of a modulator of Notch IC protease activity in the manufacture of a medicament for vaccination against a pathogen.

According to a further aspect of the invention, there is provided the use of a modulator of Notch IC protease activity in the manufacture of a medicament for vaccination against a tumour.

According to a further aspect of the invention, there is provided the use of a modulator of Notch IC protease activity in the manufacture of a medicament for increasing the immune response against a tumour or pathogen antigen or antigenic determinant.

According to a further aspect of the invention, there is provided a method for stimulating the immune system by administering a modulator of Notch IC protease activity

According to a further aspect of the invention, there is provided a method for vaccinating a subject against a tumour or pathogen by administering a modulator of Notch IC protease activity

According to a further aspect of the invention, there is provided a method for increasing an immune response of a subject against a tumour or pathogen by administering a modulator of Notch IC protease activity

According to a further aspect of the invention, there is provided a method for increasing the immune response of a subject to a tumour or pathogen antigen or antigenic determinant comprising administering an effective amount of a modulator of Notch IC protease activity simultaneously, contemporaneously, separately or sequentially with said tumour or pathogen antigen or antigenic determinant or simultaneously, contemporaneously, separately or sequentially with a polynucleotide coding for said tumour or pathogen antigen or antigenic determinant.

According to a further aspect of the invention, there is provided an adjuvant composition comprising a modulator of Notch IC protease activity.

According to a further aspect of the invention, there is provided a vaccine composition comprising such an adjuvant composition and a tumour or pathogen antigen or antigenic determinant or a polynucleotide coding for a tumour or pathogen antigen or antigenic determinant.

Suitably, the vaccine composition comprises a pathogen antigen or antigenic determinant in the form of a viral, fungal, parasitic or bacterial antigen or antigenic determinant or a polynucleotide coding for a viral, fungal, parasitic or bacterial antigen or antigenic determinant. Alternatively, the vaccine composition may comprise a tumour antigen or antigenic determinant.

According to a further aspect of the invention, there is provided a product comprising:

i) a modulator of Notch IC protease activity; and

ii) a tumour or pathogen antigen or antigenic determinant or a polynucleotide coding for a tumour or pathogen antigen or antigenic determinant;

as a combined preparation for simultaneous, contemporaneous, separate or sequential use for modulation of immune response to said tumour or pathogen antigen or antigenic determinant.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 shows a schematic representation of the Notch signalling pathway;

FIG. 2 shows a schematic representation of Notch proteins (Notch 1-4);

FIG. 3 shows a schematic representation of the Notch intracellular domain;

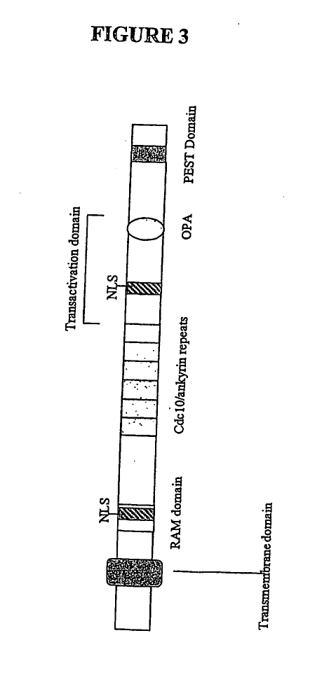

FIG. 4 shows the results of Example 1 (effect of γ-secretase inhibitor on Notch signalling in C2C12 cells transfected with mHes1-Luciferase);

FIG. 5 shows results of Example 3 (modulation of cytokine production by γ-secretase inhibitor in human CD4+ T cells); and

FIG. 6 shows results of Example 4 (effect of γ-secretase inhibitor on Delta-mediated activation of Notch signalling in Jurkat-N2 cells).

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Ir1 Press; D. M. J. Lilley and J. E. Dahlberg, 1992, Methods of Enzymology: DNA Structure Part A: Synthesis and Physical Analysis of DNA Methods in Enzymology, Academic Press; and E. M. Shevach and W. Strober, 1992 and periodic suppliements, Current Protocols in Immunology, John Wiley & Sons, New York, N.Y. Each of these general texts is herein incorporated by reference.

For the avoidance of doubt, Drosophila and vertebrate names are used interchangeably throughout the description. Both (and where applicable, all) homologues are included within the scope of the invention.

Presenilin and the Notch Signalling Pathway

As used herein, the expression “Notch signalling” is synonymous with the expression “the Notch signalling pathway” and refers to any one or more of the upstream or downstream events that result in, or from, (and including) activation of the Notch receptor.

Preferably, by “Notch signalling” we refer to any event directly upstream or downstream of Notch receptor activation or inhibition including activation or inhibition of Notch/Notch ligand interactions, upregulation or downregulation of Notch or Notch ligand expression or activity and activation or inhibition of Notch signalling transduction including, for example, proteolytic cleavage of Notch and upregulation or downregulation of the Ras-Jnk signalling pathway.

Put another way, by “Notch signalling” we refer to the Notch signalling pathway as a signal tranducing pathway comprising elements which interact, genetically and/or molecularly, with the Notch receptor protein. For example, elements which interact with the Notch protein on both a molecular and genetic basis are, by way of example only, Delta, Serrate and Deltex. Elements which interact with the Notch protein genetically are, by way of example only, Mastermind, Hairless, Su(H) and Presenilin.

In one aspect, Notch signalling includes signalling events taking place extracellularly or at the cell membrane. In a further aspect, it includes signalling events taking place intracellularly, for example within the cell cytoplasm or within the cell nucleus.

A very important component of the Notch signalling pathway is Notch receptor/Notch ligand interaction. Thus Notch signalling may involve changes in expression, nature, amount or activity of Notch ligands or receptors or their resulting cleavage products. In addition, Notch signalling may involve changes in expression, nature, amount or activity of Notch signalling pathway G-proteins or Notch signalling pathway enzymes such as proteases, kinases (e.g. serine/threonine kinases), phosphatases, ligases (e.g. ubiquitin ligases) or glycosyltransferases. Alternatively Notch signalling may involve changes in expression, nature, amount or activity of DNA binding elements such as transcription factors.

In a preferred form of the invention Notch signalling means specific signalling, meaning that the signal detected results substantially or at least predominantly from the Notch signalling pathway, and preferably from Notch/Notch ligand interaction, rather than any other significant interfering or competing cause, such as cytokine signalling. The Notch signalling pathway is described in more detail below.

Notch signalling directs binary cell fate decisions in the embryo. Notch was first described in Drosophila as a transmembrane protein that functions as a receptor for two different ligands, Delta and Serrate. Vertebrates express multiple Notch receptors and ligands. At least four Notch receptors (Notch-1, Notch-2, Notch-3 and Notch-4) have been identified to date in human cells.

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an C-terminal fragment consisting of a portion of the extracellular domain, the transmembrane domain and the intracellular domain, and the other comprising the majority of the extracellular domain. Activation of Notch signalling involves proteolytic cleavage of the extracellular domain, involving TNF convertase (TACE), and intramembranous cleavage by presenilin-dependent γ-secretase activity.

Notch receptors are inserted into the membrane as disulphide-linked heterodimeric molecules consisting of an extracellular domain containing up to 36 epidermal growth factor (EGF)-like repeats and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and, like the ankyrin-like repeats, is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas).

The Notch receptor is activated by binding of extracellular ligands, such as Delta (Delta 1, 3 or 4), Serrate (Serrate 1 or 2 or their homologues Jagged 1 and 2) and Scabrous, to the EGF-like repeats of Notch's extracellular domain. Delta may require cleavage for activation. It is cleaved by the ADAM disintegrin metalloprotease Kuzbanian at the cell surface, the cleavage event releasing a soluble and active extracellular fragment of Delta. An oncogenic variant of the human Notch-1 protein, also known as TAN-1, which has a truncated extracellular domain, is constitutively active and has been found to be involved in T cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm. Upon interaction of the Notch receptor with its ligand Delta on adjacent cells Su(H) disassociates from the Notch intracellular domain, where it is replaced by Deltex, and translocates into the nucleus. Su(H) interacts with responsive elements found in the promoters of several genes and has been found to be a critical downstream protein in the Notch signalling pathway. Target genes of Su(H) and of Notch signalling in general are listed below. The involvement of Su(H) in transcription is thought to be modulated by Hairless.

As noted above the intracellular domain of Notch (NotchIC) also has a direct nuclear function (Lieber). Recent studies have indeed shown that Notch activation generally requires that the six cdc10/ankyrin repeats of the Notch intracellular domain reach the nucleus and participate in transcriptional activation. The site of proteolytic cleavage on the intracellular tail of Notch has been identified between gly1743 and val 1744 (termed site 3, or S3) (Schroeter). It is thought that the proteolytic cleavage step that releases the NotchIC for nuclear entry is dependent on Presenilin activity.

The intracellular domain has been shown to accumulate in the nucleus where it forms a transcriptional regulator complex with other transcription factors such as the CSL family member CBF1 (suppressor of hairless, Su(H) in Drosophila, Lag-2 in C. elegans) (Schroeter; Struhl) and Mastermind (MAML1/2). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

NotchIC processing occurs in response to binding of Notch ligands Delta or Serrate/Jagged. The post-translational modification of the nascent Notch receptor in the Golgi (Munro; Ju) appears, at least in part, to control which of the two types of ligand it interacts with on a cell surface. The Notch receptor is modified on its extracellular domain by Fringe, a glycosyl transferase enzyme that binds to the Notch/Lin motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially interact with Delta (Panin; Hicks). Although Drosophila has a single Fringe gene, vertebrates are known to express multiple genes (Radical, Manic and Lunatic Fringes) (Irvine).

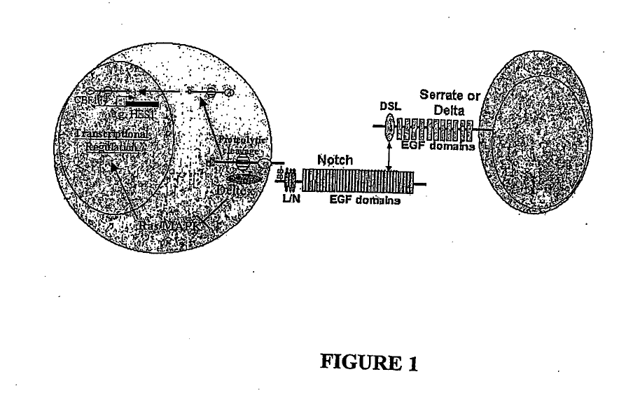

Thus, signal transduction from the Notch receptor can occur via different pathways (FIG. 1). The better defined pathway involves proteolytic cleavage of the intracellular domain of Notch (NotchIC) that translocates to the nucleus and forms a transcriptional activator complex with the CSL family protein CBF1 (supressor of hairless, Su(H) in Drosophila, Lag-2 in C. elegans). NotchIC-CBF1 complexes then activate target genes, such as Deltex or the bHLH proteins HES (hairy-enhancer of split like) 1 and 5. Notch can also signal in a CBF1-independent manner that involves the cytoplasmic zinc finger containing protein Deltex (FIG. 1). Unlike CBF1, Deltex does not move to the nucleus following Notch activation. Instead, it interacts with Grb2 and modulates the Ras-Jnk signalling pathway which, in turn, modulates transcription of target genes.

Presenilins are integral membrane proteins with seven to eight transmembrane domains and a hydrophilic loop located between the transmembrane domains 6 and 7. Two presenilin genes have so far been identified: PS1 and PS2. More than 60% of amino acid residues in the sequence of PS1 and PS2 are conserved. The two proteins share major structural similarities, tissue-specific alternative splicing patterns and predicted tertiary structure. The presenilin genes have been identified as major causal genes for early onset familial Alzheimer's disease (FAD). FAD mutations are found throughout the entire PS1 molecule. However, two intramembranous aspartates at residues 257 and 385 have been revealed to be critical to the proper functioning of the protein (Capell et al).

Non-human homologues of the PS1 and PS2 genes and proteins have now been identified, isolated and cloned. Amongst them are the murine homologue (PS1) of human PS1, a C. elegans member (SEL-12) and a D. melangoster member (DmPS) of the presenilin gene family. Each of these genes and proteins have been identified on the basis of their high degrees of homology to the PS1/PS2 genes. Modulators of any of these genes and proteins, or any others which are known or become available, are included in the scope of the present invention.

Modulators of Notch IC Protease Activity

The term “modulate” as used herein in relation to Notch IC protease activity in general and presenilin or presenilin-dependent gamma-secretase activity in particular refers to a change or alteration in the biological activity of enzymes responsible for cleaving Notch to release Notch IC. Thus, modulation of Notch IC protease activity includes inhibition or down-regulation of Notch signalling, e.g. by compounds which block, at least to some extent, the normal biological activity of such enzymes. Alternatively, the term “modulation” may refer to the activation or up-regulation of Notch IC protease activity, e.g. by compounds which stimulate or upregulate, at least to some extent, the normal biological activity of such enzymes.

Preferably the modulator of Notch IC protease activity is a modulator of Notch IC protease activity such as presenilin, a presenilin-dependent gamma-secretase or a presenilin-dependent gamma-secretase complex.

In other words, modulators of presenilin or presenilin-dependent gamma-secretase include compounds capable of activating or inhibiting the expression and/or activity of presenilin or presenilin-dependent gamma-secretase.

Presenilin or Presenilin-Dependent Gamma-Secretase Activators

By a compound capable of activating presenilin or presenilin-dependent gamma-secretase, we refer to compounds capable of activating any one or more of the polypeptides or polynucleotides of the presenilin or presenilin-dependent gamma-secretase family, in particular PS1 and PS2, and any homologues, derivatives or variants thereof.

In one embodiment, the molecule for presenilin or presenilin-dependent gamma-secretase activation will be a dominant negative version of a presenilin or presenilin-dependent gamma-secretase repressor, respectively. In an alternative embodiment, the molecule for presenilin or presenilin-dependent gamma-secretase activation will be capable of inhibiting a presenilin or presenilin-dependent gamma-secretase repressor, respectively. In a further alternative embodiment, the molecule for presenilin or presenilin-dependent gamma-secretase activation will be a positive activator of presenilin or presenilin-dependent gamma-secretase, respectively.

In a particular embodiment, the molecule will be capable of inducing or increasing presenilin or presenilin-dependent gamma-secretase expression. Such a molecule may be a nucleic acid sequence capable of inducing or increasing presenilin or presenilin-dependent gamma-secretase expression.

In one embodiment, the molecule will be capable of up-regulating expression of endogenous presenilin or presenilin-dependent gamma-secretase in target cells. In particular, the molecule may be an immunosuppressive cytokine capable of up-regulating the expression of endogenous presenilin or presenilin-dependent gamma-secretase in target cells, or a polynucleotide which encodes such a cytokine. Immunosuppressive cytokines include IL-4, IL-10, IL-13, TGF-β and FLT3 ligand.

Preferably, the molecule will be a polypeptide selected from polypeptides of the ALG family, in particular ALG-3, Nicastrin, Calsenilin, β-catenin or Bcl-X(L), or variants, derivatives or fragments thereof or a polynucleotide encoding such a polypeptide or a variant, derivative or fragment thereof.

ALG-3 is a mouse homologue of the Chromosome 1 familial Alzheimer's disease gene PS2. It codes for a truncated PS2 polypeptide (the 103 COOH-terminal PS2 amino-acids) that is capable of inhibiting the apoptotic role of PS2 (D'Adamino et al). It has indeed been found that PS2 is required for some forms of cell death in diverse cell types and that ALG-3 rescues mouse T hybridoma 3DO cells from T cell receptor-induced apoptosis by inhibiting Fas ligand induction and Fas signalling (Lacana et al). ALG-3 has also been shown to reduce protease activity and to antagonise polymerase cleavage upon Fas triggering. The polynucleotide sequence of ALG-3 can be found at GenBank Accession Number U49111.

Nicastrin is a type 1 transmembrane glycoprotein which has a domain found in the aminopeptidase/transferrin receptor superfamily (Fagan et al). It acts as a key regulator for presenilin-mediated gamma-secretase cleavage of ∃-amyloid precursor protein by forming a functional complex with PS1 and PS2. It plays a central role in presenilin mediated processing of Notch (Gang et al). Suppression of Nicastrin expression in C. elegans embryos induces a subset of Notch phenotypes similar to those induced by simultaneous null mutations in both presenilin homologues of C. elegans. Thus, it is thought that Nicastrin and presenilins are functional components of a multimeric complex necessary for the intermembranous proteolysis of the Notch protein. The polynucleotide sequence of Nicastrin can be found at GenBank Accession Numbers NM_{—}021607 (Mus musculus), AF240470 (Drosophila melanogaster) and AF240468 (Homo sapiens).

Bcl-X(L), an anti-apoptotic member of the Bcl-2 family, has been shown to interact with the carboxyl-terminal fragments of PS1 and PS2 (Passer et al). Furthermore, it has been demonstrated that Bcl-X(L) and PS2 partially co-localise to sites of the vesicular transport system. The polynucleotide sequence of Bcl-X(L) can be found at GenBank Accession Number NM_{—}004050.

Calsenilin is a member of the recoverin family of neuronal calcium binding proteins that have been shown to interact with PS1 and PS2. Calsenilin has the ability to interact with the endogenous 25-kDa presenilin C-terminal fragment that is produced by regulated endoproteolytic cleavage. Thus, the expression of calsenilin can regulate levels of an active proteolytic product of presenilin (Choi et al). The polynucleotide sequence of calsenilin can be found at GenBank Accession Numbers NM_{—}032462 (Rattus norvegicus), XM_{—}015414 (Homo sapiens) and NM_{—}019789 (Mus musculus).

Beta-catenin binds PS1 in an interaction thought to mechanistic in Alzheimer's disease. The cyclin-dependent kinase p35/cdk5 binds and phosphorylates beta-catenin thus regulating its interaction with PS1 (Kesavapany et al). The polynucleotide sequence of Xenopus laevis beta-catenin can be found at GenBank Accession Number M77013.

In a preferred embodiment, the activator will be a constitutively active presenilin or presenilin-dependent gamma-secretase or a homologue, variant, derivative or fragment thereof or a polynucleotide encoding such a presenilin. Alternatively, the activator may be a molecular mimic of a constitutively active presenilin or presenilin-dependent gamma-secretase.

By polypeptides or polynucleotides for presenilin or presenilin-dependent gamma-secretase activation, we also include molecules activated or expressed as a result of presenilin or presenilin-dependent gamma-secretase activation and any compounds involved in the activation or expression of such molecules. Examples of such molecules are the Notch Intracellular Domain (NICD), the CSL family protein CBF1 (Su(H) in Drosophila, Lag-2 in C. elegans), bHLH proteins HES1 and HES5.

Activation of presenilin or presenilin-dependent gamma-secretase may also be achieved by repressing inhibitors of presenilin or presenilin-dependent gamma-secretase, respectively. As such, presenilin or presenilin-dependent gamma-secretase activators include molecules capable of repressing any presenilin or presenilin-dependent gamma-secretase inhibitors, respectively. Preferably, the molecule will be a polypeptide, or a polynucleotide encoding such a polypeptide, that decreases or interferes with the production or activity of compounds that are capable of producing a decrease in the expression or activity of presenilin or presenilin-dependent gamma-secretase. In a preferred embodiment, the molecules will be capable of repressing polypeptides such as 26S proteasome.

Presenilin or Presenilin-Dependent Gamma-Secretase Inhibitors

By a polypeptide capable of inhibiting presenilin or presenilin-dependent gamma-secretase, we mean a molecule capable of inhibiting any one or more of the polypeptides or polynucleotides of the presenilin or presenilin-dependent gamma-secretase family, in particular PS1 and PS2, and any homologues, derivatives or variants thereof.

In one embodiment, the molecule for presenilin or presenilin-dependent gamma-secretase inhibition will be a dominant negative version of a presenilin or presenilin-dependent gamma-secretase activator, respectively. In an alternative embodiment, the molecule for presenilin or presenilin-dependent gamma-secretase inhibition will be capable of inhibiting a presenilin or presenilin-dependent gamma-secretase activator, respectively. In a further alternative embodiment, the molecule for presenilin or presenilin-dependent gamma-secretase activation will be a direct repressor of presenilin or presenilin-dependent gamma-secretase, respectively.

In a particular embodiment, the molecule will be capable of reducing or preventing presenilin or presenilin-dependent gamma-secretase expression. Such a molecule may be a nucleic acid sequence capable of reducing or preventing presenilin or presenilin-dependent gamma-secretase expression.

In one embodiment, the molecule will be capable of down-regulating expression and/or activity of endogenous presenilin or presenilin-dependent gamma-secretase in target cells. Preferably, the molecule will be a polypeptide selected from polypeptides of the proteasome family, in particular 26S proteasome or Sel 10 and its mammalian homologues, or variants, derivatives or fragments thereof or a polynucleotide encoding such a polypeptide or a variant, derivative or fragment thereof.

26S proteasome is capable of degrading PS1 by causing endoproteolytic cleavage of the protein near residue 298 (Fraser et al).

In another embodiment, the inhibitor will be a polypeptide, or a polynucleotide encoding such a polypeptide, that decreases or interferes with the production of compounds that are capable of producing an increase in the expression of presenilin or presenilin-dependent gamma-secretase.

Alternatively, the inhibitor is a polynucleotide, preferably an antisense construct derived from a sense nucleotide sequence encoding a polypeptide selected from presenilin or presenilin-dependent gamma-secretase and presenilin or presenilin-dependent gamma-secretase activators such as ALG-3, Nicastrin, Calsenilin, β-catenin or Bcl-X(L), derivatives, fragments, variants and homologues thereof.

Inhibitors of presenilin further include compounds capable of repressing the expression or activity of molecules normally activated by the expression or activity of presenilin or presenilin-dependent gamma-secretase (e.g. CBF1, HES1 or HES5) and compounds the expression or activity of which is normally repressed by that of presenilin or presenilin-dependent gamma-secretase. Proteins for presenilin or presenilin-dependent gamma-secretase inhibition will also include variants of the above-described activators of presenilin or presenilin-dependent gamma-secretase which have been modified in such a way as to block rather than activate or transduce presenilin or presenilin-dependent gamma-secretase. An example of such an inhibitor would be a presenilin protein or presenilin-dependent gamma-secretase modified in such a way that it binds to but does not cleave Notch.

Modulators of the Notch Signalling Pathway

In a preferred embodiment of the present invention, the modulator of presenilin or presenilin-dependent gamma-secretase is used in conjunction with a modulator of the Notch signalling pathway, i.e. a compound capable of up-regulating or down-regulating the Notch signalling pathway.

The term “modulate” as used herein refers to a change or alteration in the biological activity of the Notch signalling pathway or a target signalling pathway thereof. Thus, modulation of Notch signalling includes inhibition or down-regulation of Notch signalling, e.g. by compounds which block, at least to some extent, the normal biological activity of the Notch signalling pathway. Alternatively, the term “modulation” may refer to the activation or up-regulation of Notch signalling, e.g. by compounds which stimulate or upregulate, at least to some extent, the normal biological activity of the Notch signalling pathway. “Immunomodulation” refers to a change or alteration in the function of the immune system.

Up-Regulators of the Notch Signalling Pathway

Compounds capable of up-regulating the Notch signalling pathway are compounds capable of transducing or activating the Notch signalling pathway. By a polypeptide or polynucleotide which is for Notch signalling transduction we include a molecule which participates in signalling through Notch receptors including activation of Notch, the downstream events of the Notch signalling pathway, transcriptional regulation of downstream target genes and other non-transcriptional downstream events (e.g. post-translational modification of existing proteins). More particularly, the second sequence is a domain that allows activation of target genes of the Notch signalling pathway, or a polynucleotide sequence which codes therefor.

In other words, by modulating Notch signalling transduction we include:

a) activation of the Notch signalling pathway by (i) dominant negative or inhibitors of repressors and (ii) activators; and

b) blockade of the Notch signalling pathway by (i) dominant negative or inhibitors of activators and (ii) inhibitors.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H) to contribute an activation domain that allows Su(H) to activate the transcription of E(spl) as well as other target genes. It should also be noted that Su(H) is not required for all Notch-dependent decisions, indicating that Notch mediates some cell fate choices by associating with other DNA-binding transcription factors or by employing other mechanisms to transduce extracellular signals.

According to one aspect of the present invention the second sequence is the Notch polypeptide or polynucleotide or a fragment thereof which retains the signalling transduction ability of Notch or an analogue of Notch which has the signalling transduction ability of Notch. By Notch, we mean Notch-1, Notch-2, Notch-3, Notch-4 and any other Notch homologues or analogues. In a particularly preferred embodiment the second amino acid sequence is the Notch intracellular domain (Notch IC) or a sub-fragment thereof.

As used herein the term “analogue of Notch” includes variants thereof which retain the signalling transduction ability of Notch. By “analogue” we include a protein which has Notch signalling transduction ability, but generally has a different evolutionary origin to Notch. Analogues of Notch include proteins from the Epstein Barr virus (EBV), such as EBNA2, BARF0 or LMP2A.

By a polypeptide or polynucleotide which is for Notch signalling activation we mean a molecule which is capable of activating Notch, the Notch signalling pathway or any one or more of the components of the Notch signalling pathway.

In one embodiment, the molecule for Notch signalling activation will be a dominant negative version of a Notch signalling repressor. In an alternative embodiment, the molecule for Notch signalling activation will be capable of inhibiting a Notch signalling repressor. In a further alternative embodiment, the molecule for Notch signalling activation will be a positive activator of Notch signalling.

In a particular embodiment, the molecule will be capable of inducing or increasing Notch or Notch ligand expression. Such a molecule may be a nucleic acid sequence capable of inducing or increasing Notch or Notch ligand expression.

In one embodiment, the molecule will be capable of up-regulating expression of the endogenous genes encoding Notch or Notch ligands in target cells. In particular, the molecule may be an immunosuppressive cytokine capable of up-regulating the expression of endogenous Notch or Notch ligands in target cells, or a polynucleotide which encodes such a cytokine. Immunosuppressive cytokines include IL-4, IL-10, IL-13, TGF-beta and FLT3 ligand.

Preferably, the molecule will be a polypeptide selected from Noggin, Chordin, Follistatin, Xnr3, fibroblast growth factors and derivatives, fragments, variants and homologues thereof, or a polynucleotide encoding any one or more of the above.

In another embodiment, the molecule may be a Notch ligand, or a polynucleotide encoding a Notch ligand. Notch ligands of use in the present invention include endogenous Notch ligands which are typically capable of binding to a Notch receptor polypeptide present in the membrane of a variety of mammalian cells, for example hemapoietic stem cells.

Particular examples of mammalian Notch ligands identified to date, and of use in the present invention, include the Delta family, for example Delta (Genbank Accession No. AF003522-Homo sapiens), Delta-3 (Genbank Accession No. AF084576-Rattus norvegicus) and Delta-like 3 (Mus musculus), and the Serrate family, for example Serrate-1 and Serrate-2 (WO97/01571, WO96/27610 and WO92/19734), Jagged-1 and Jagged-2 (Genbank Accession No. AF029778-Homo sapiens), and LAG-2. Homology between family members is extensive. For example, human Jagged-2 has 40.6% identity and 58.7% similarity to Serrate.

In a preferred embodiment, the activator will be a constitutively active Notch receptor or Notch intracellular domain, or a polynucleotide encoding such a receptor or intracellular domain.

In an alternative embodiment, the activator of Notch signalling will act downstream of the Notch receptor. Thus, for example, the activator of Notch signalling may be a constitutively active Deltex polypeptide or a polynucleotide encoding such a polypeptide. Other downstream components of the Notch signalling pathway of use in the present invention include Deltex-1, Deltex-2, Deltex-3, Suppressor of Deltex (SuDx), Numb and isoforms thereof, Numb associated Kinase (NAK), Notchless, Dishevelled (Dsh), emb5, Fringe genes (such as Radical, Lunatic and Manic), Fringe Connection PON, LNX, Disabled, Numblike, Nur77, NFkB2, Mirror, Warthog, Engrailed-1 and Engrailed-2, Lip-1 and homologues thereof, the polypeptides involved in the Ras/MAPK cascade modulated by Deltex, polypeptides involved in the proteolytic cleavage of Notch such as Presenilin and polypeptides involved in the transcriptional regulation of Notch target genes, preferably in a constitutively active form, and analogues, derivatives, variants and fragments thereof.

By polypeptides or polynucleotides for Notch signalling activation is also meant any polypeptides expressed as a result of Notch activation and any polypeptides involved in the expression of such polypeptides, or polynucleotides encoding for such polypeptides.

Activation of Notch signalling may also be achieved by repressing inhibitors of the Notch signalling pathway. As such, polypeptides for Notch signalling activation will include molecules capable of repressing any Notch signalling inhibitors. Preferably the molecule will be a polypeptide, or a polynucleotide encoding such a polypeptide, that decreases or interferes with the production or activity of compounds that are capable of producing a decrease in the expression or activity of Notch, Notch ligands, or any downstream components of the Notch signalling pathway. In a preferred embodiment, the molecules will be capable of repressing polypeptides of the Toll-like receptor protein family, cytokines such as IL-12, IFN-γ, TNF-α, and growth factors such as the bone morphogenetic protein (BMP), BMP receptors and activins, derivatives, fragments, variants and homologues thereof.

Notch Ligand Domains

As discussed above, Notch ligands typically comprise a number of distinctive domains. Some predicted/potential domain locations for various naturally occurring human Notch ligands (based on amino acid numbering in the precursor proteins) are shown below:

Human Delta 1

| Component | Amino acids | Proposed function/domain |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| Component | Amino acids | Proposed function/domain |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| Component | Amino acids | Proposed function/domain |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| Component | Amino acids | Proposed function/domain |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| Component | Amino acids | Proposed function/domain |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence:

| | |
|---|---|
| Cys Xaa Xaa Xaa Xaa Xaa Xaa Xaa Xaa Cys Xaa Xaa Xaa Cys Xaa Xaa | (SEQ ID NO:7) |
| Xaa Xaa Xaa Xaa Xaa Xaa Xaa Xaa Xaa Cys Xaa Xaa Xaa Xaa Xaa Xaa | |
| Xaa Cys Xaa Xaa Xaa Xaa Xaa Xaa Xaa Xaa Cys | |

Preferably the DSL domain may include most or all of the following consensus amino acid sequence:

| | |
|---|---|
| Cys Xaa Xaa Xaa ARO ARO Xaa Xaa Xaa Cys Xaa Xaa Xaa Cys BAS NOP | (SEQ ID NO:8) |
| BAS ACM ACM Xaa ARO NOP ARO Xaa Xaa Cys Xaa Xaa Xaa NOP Xaa Xaa | |
| Xaa Cys Xaa Xaa NOP ARO Xaa NOP Xaa Xaa Cys | |

wherein:

ARO is an aromatic amino acid residue, such as tyrosine, phenylalanine, tryptophan or histidine;

NOP is a non-polar amino acid residue such as glycine, alanine, proline, leucine, isoleucine or valine;

BAS is a basic amino acid residue such as arginine or lysine; and

ACM is an acid or amide amino acid residue such as aspartic acid, glutamic acid, asparagine or glutamine.

Preferably the DSL domain may include most or all of the following consensus amino acid sequence:

| | |
|---|---|
| Cys Xaa Xaa Xaa Tyr Tyr Xaa Xaa Xaa Cys Xaa Xaa Xaa Cys Arg Pro | (SEQ ID NO:9) |
| Arg Asx Asp Xaa Phe Gly His Xaa Xaa Cys Xaa Xaa Xaa Gly Xaa Xaa | |
| Xaa Cys Xaa Xaa Gly Trp Xaa Gly Xaa Xaa Cys | |

(wherein Xaa may be any amino acid and Asx is either aspartic acid or asparagine).

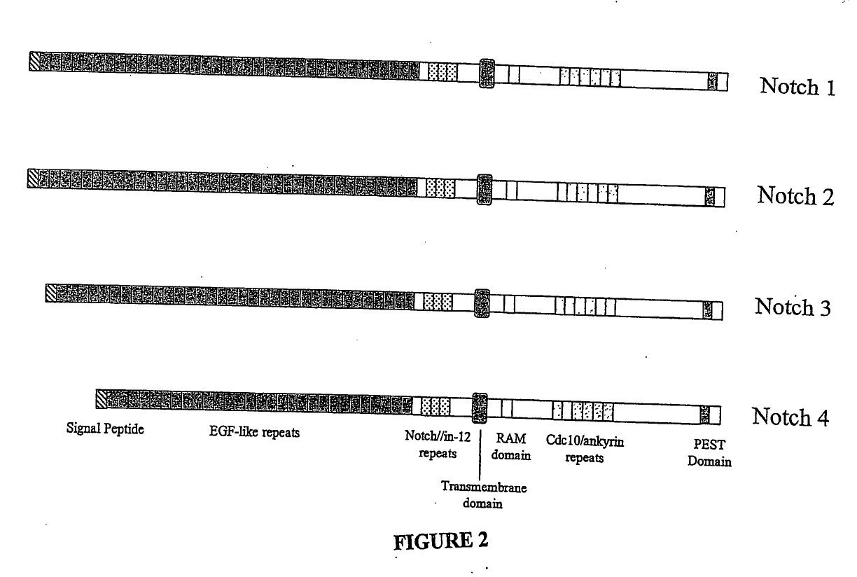

An alignment of DSL domains from Notch ligands from various sources is shown in FIG. 2.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

It will be appreciated that the term “DSL domain” as used herein includes sequence variants, fragments, derivatives and mimetics having activity corresponding to naturally occurring domains.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors 1X and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE a typical EGF domain may include six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain:

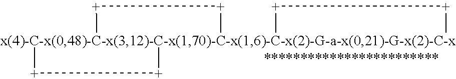

wherein:

‘C’: conserved cysteine involved in a disulfide bond.

‘G’: often conserved glycine

‘a’: often conserved aromatic amino acid

‘*’: position of both patterns.

‘x’: any residue

The region between the 5th and 6th cysteines contains two conserved glycines, at least one of which is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

It will be appreciated that the term “EGF domain” as used herein includes sequence variants, fragments, derivatives and mimetics having activity corresponding to naturally occurring domains.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X % identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity.

Down-Regulators of Notch Signalling Pathway

By a polypeptide or polynucleotide which is for Notch signalling inhibition, we mean a molecule which is capable of inhibiting Notch, the Notch signalling pathway or any one or more of the components of the Notch signalling pathway.

In one embodiment, the molecule for Notch signalling inhibition will be a dominant negative version of a compound capable of activating or transducing Notch signalling. In an alternative embodiment, the molecule for Notch signalling inhibition will be capable of repressing a compound capable of activating or transducing Notch signalling. In a further alternative embodiment, the molecule for Notch signalling inhibition will be an inhibitor of Notch signalling.

In a particular embodiment, the molecule will be capable of reducing or preventing Notch or Notch ligand expression. Such a molecule may be a nucleic acid sequence capable of reducing or preventing Notch or Notch ligand expression.

Preferably the nucleic acid sequence encodes a polypeptide selected from Toll-like receptor protein family, a cytokine such as IL-12, IFN-γ, TNF-α, or a growth factor such as a bone morphogenetic protein (BMP), a BMP receptor and activins. Preferably the agent is a polypeptide, or a polynucleotide encoding such a polypeptide, that decreases or interferes with the production of compounds that are capable of producing an increase in the expression of Notch ligand, such as Noggin, Chordin, Follistatin, Xnr3, fibroblast growth factors and derivatives, fragments, variants and homologues thereof.

Alternatively, the nucleic acid sequence is an antisense construct derived from a sense nucleotide sequence encoding a polypeptide selected from a Notch ligand and a polypeptide capable of up-regulating Notch ligand expression, such as Noggin, Chordin, Follistatin, Xnr3, fibroblast growth factors and derivatives, fragments, variants and homologues thereof.

In another preferred embodiment the inhibitor of Notch signalling is a molecule which is capable of modulating Notch-Notch ligand interactions. A molecule may be considered to modulate Notch-Notch ligand interactions if it is capable of inhibiting the interaction of Notch with its ligands, preferably to an extent sufficient to provide therapeutic efficacy.

In this embodiment the molecule may be a polypeptide, or a polynucleotide encoding such a polypeptide, selected from a Toll-like receptor, a cytokine such as IL-12, IFN-γ, TNF-α, or a growth factor such as a BMP, a BMP receptor and activins. Preferably the polypeptide decreases or interferes with the production of an agent that is capable of producing an increase in the expression of Notch ligand, such as Noggin, Chordin, Follistatin, Xnr3, fibroblast growth factors and derivatives, fragments, variants, homologues and analogs thereof.

Preferably when the inhibitor is a receptor or a nucleic acid sequence encoding a receptor, the receptor is activated. Thus, for example, when the agent is a nucleic acid sequence, the receptor is constitutively active when expressed.

Inhibitors of Notch signalling also include downstream inhibitors of the Notch signalling pathway (such as Dsh and Numb), compounds that prevent expression of Notch target genes or induce expression of genes repressed by the Notch signalling pathway and dominant negative versions of Notch signalling transducer molecules (such as of Notch IC and Deltex). Proteins for Notch signalling inhibition will also include variants of the wild-type components of the Notch signalling pathway which have been modified in such a way that their presence blocks rather than transduces the signalling pathway. An example of such a compound would be a Notch receptor which has been modified such that proteolytic cleavage of its intracellular domain is no longer possible.

Cells of Use in the Invention

Cells of use in the present invention may be tumour cells or cells of the immune system and will be capable of transducing the Notch signalling pathway.

Tumour Cells Expressing Notch Ligand

The expression of Notch ligands in melanoma cell lines has been identified. Other tumour cells may also be tested for expression of Notch ligands using a variety of techniques known in the art such as detection of mRNA by RT-PCR or detection of the Notch ligand polypeptides by Western blotting. Suitable tumour cells to be tested include cells present in malignancies such as cancer of the breast, cervix, colon, rectum, endometrium, kidney, lung, ovary, pancreas, prostate gland, skin, stomach, bladder, CNS, oesophagus, head-or-neck, liver, testis, thymus or thyroid. Malignant blood cells, bone marrow cells, B-lymphocytes, T-lymphocytes, lymphocytic progenitors or myeloid cell progenitors may also be tested.

Tumour cells which express Notch ligand may be a tumour cells from a solid tumour or a non-solid tumour and may be a primary tumour cell or a disseminated metastatic (secondary) tumour cell. Non-solid tumours include myeloma; leukaemia (acute or chronic, lymphocytic or myelocytic) such as acute myeloblastic, acute promyelocytic, acute myelomonocytic, acute monocytic, erythroleukaemia; and lymphomas such as Hodgkin's, non-Hodgkin's and Burkitt's. Solid tumours include carcinoma, colon carcinoma, small cell lung carcinoma, non-small cell lung carcinoma, adenocarcinoma, melanoma, basal or squamous cell carcinoma, mesothelioma, adenocarcinoma, neuroblastoma, glioma, astrocytoma, medulloblastoma, retinoblastoma, sarcoma, osteosarcoma, rhabdomyosarcoma, fibrosarcoma, osteogenic sarcoma, hepatoma, and seminoma.

Antigen Presenting Cells

Antigen-presenting cells (APCs) for use in the present invention may be “professional” antigen presenting cells or may be another cell that may be induced to present antigen to T cells. Alternatively a APC precursor may be used which differentiates or is activated under the conditions of culture to produce an APC. An APC for use in the ex vivo methods of the invention is typically isolated from a tumour or peripheral blood found within the body of a patient. Preferably the APC or precursor is of human origin. However, where APCs are used in preliminary in vitro screening procedures to identify and test suitable nucleic acid sequences, APCs from any suitable source, such as a healthy patient, may be used.

APCs include dendritic cells (DCs) such as interdigitating DCs or follicular DCs, Langerhans cells, PBMCs, macrophages, B-lymphocytes, T-lymphocytes, or other cell types such as epithelial cells, fibroblasts or endothelial cells, activated or engineered by transfection to express a MHC molecule (Class I or II) on their surfaces. Precursors of APCs include CD34⁺ cells, monocytes, fibroblasts and endothelial cells. The APCs or precursors may be modified by the culture conditions or may be genetically modified, for instance by transfection of one or more genes encoding proteins which play a role in antigen presentation and/or in combination of selected cytokine genes which would promote to immune potentiation (for example IL-2, IL-12, IFN-γ, TNF-α, IL-18 etc.). Such proteins include MHC molecules (Class I or Class II), CD80, CD86, or CD40. Most preferably DCs or DC-precursors are included as a source of APCs.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba et al., 1992), or from bone marrow, non-adherent CD34⁺ cells can be treated with GM-CSF and TNF-α (Caux et al., 1992). DCs can also be routinely prepared from the peripheral blood of human volunteers, similarly to the method of Sallusto and Lanzavecchia (1994) using purified peripheral blood mononucleocytes (PBMCs) and treating 2 hour adherent cells with GM-CSF and IL-4. If required, these may be depleted of CD 19⁺ B cells and CD3⁺, CD2⁺ T cells using magnetic beads (see Coffin et al., 1998). Culture conditions may include other cytokines such as GM-CSF or IL-4 for the maintenance and, or activity of the dendritic cells or other antigen presenting cells.

T cells

Where T cells are to be used in the ex vivo methods of the invention, the T cells are typically infiltrating T lymphocytes isolated from a solid tumour within the body of an individual suffering from a cancer. Alternatively other T cells such as CD8⁺ cells may be used. It may also be convenient to use cell lines such as T cell hybridomas. However, where T cells are used in preliminary in vitro screening procedures to identify and test suitable nucleic acid sequences, T cells from any suitable source, such as a healthy patient, may be used and may be obtained from blood or another source (such as lymph nodes, spleen, or bone marrow). They may optionally be enriched or purified by standard procedures. The T cells may be used in combination with other immune cells, obtained from the same or a different individual. Alternatively whole blood may be used or leukocyte enriched blood or purified white blood cells as a source of T cells and other cell types. It is particularly preferred to use helper T cells (CD4⁺).

Lymphocytes with antigen receptors recognising antigens presented by tumour cells (tumour-reactive lymphocytes (TRLs)) can be isolated from peripheral blood, lymph nodes or from tumour tissue (tumour-infiltrating lymphocytes (TILs)). Methods for isolating and culturing TRLs are well known in the art. See for example Vose et al. (1977). TILs and other TRLs may be isolated and expanded in culture in the presence of cytokines such as Interleukin (IL)-2, IL-12, IFN-γ, TNF-α, IL-18 as described by Belldegrun et al. (1988); Belldegrun et al. (1989); and Spiess et al (1987). TRLs and TILs reactive with identified tumour antigens can also be isolated using MHC Class-I and Class-II tetramer technology (Dunbar et al., 1998; Romero et al., 1998).

Thus, it will be understood that the term “antigen presenting cell or the like” are used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

Assays

In one embodiment of the present invention, small molecules may be screened for their ability to bind presenilin or presenilin-containing complexes or components or presenilin-containing complexes, especially human PS1 or PS2. In another embodiment, compounds may be tested for their ability to induce or repress expression of presenilin, especially human PS1 or PS2. In another embodiment they may be tested for their ability to induce or repress activity of presenilin-dependent γ-secretase. Synthetic peptide substances, including for example those derived from targets of presenilin-dependent γ-secretase activity such as amyloid precursor protein (APP) may be used in assays to detect modulators. These and other embodiments are described below.

Identification of Presenilin or Presenilin-Dependent Gamma-Secretase Modulators

The assay of the present invention is set up to detect either inhibition or enhancement of presenilin or presenilin-dependent gamma-secretase expression and/or activity in cells of the immune system by candidate compounds. The compounds may be small molecules, proteins, antibodies or other ligands. Amounts or activity of presenilin or presenilin-dependent gamma-secretase will be measured for each compound tested using standard assay techniques and appropriate controls. Preferably the detected signal is compared with a reference signal and any modulation with respect to the reference signal measured. The assay may also be run in the presence of a known antagonist of presenilin or presenilin-dependent gamma-secretase in order to identify compounds capable of rescuing Notch IC protease activity and/or expression, respectively.

Expression and/or activity of presenilin or presenilin-dependent gamma-secretase will be measured in proportion to cleavage of Notch Intracellular Domain (NICD) or in proportion to levels of activity or expression of downstream components of the NICD signalling pathway. Such components will be referred to as “targets” of the NICD pathway. Known targets include Deltex, Hes-1, E(spl), IL-10, CD-23, Dlx-1, CTLA4, CD-4, Numb, Mastermind and Dsh.

Any one or more of appropriate targets—such as an amino acid sequence and/or nucleotide sequence—may be used for identifying a compound capable of modulating Notch IC protease activity and/or expression in cells of the immune system in any of a variety of drug screening techniques. The target employed in such a test may be free in solution, affixed to a solid support, borne on a cell surface, or located intracellularly.

The assay of the present invention may be a screen, whereby a number of agents are tested. In one aspect, the assay method of the present invention is a high throughput screen.

Techniques for drug screening may be based on the method described in Geysen, European Patent Application 84/03564, published on Sep. 13, 1984. In summary, large numbers of different small peptide test compounds are synthesised on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO-A-84/03564.

It is expected that the assay methods of the present invention will be suitable for both small and large-scale screening of test compounds as well as in quantitative assays.

Various nucleic acid assays are known. Any conventional technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

PCR technology as described e.g. in section 14 of Sambrook et al., 1989, requires the use of oligonucleotide probes that will hybridise to nucleic acid. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating α³²P dATP with the Klenow fragment of DNA polymerase in a random priming reaction, as is well known in the art. Oligonucleotides are usually end-labelled with γ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

Preferred are such sequences, probes which hybridise under high-stringency conditions.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na+ at 65-68° C. High stringency conditions can be provided, for example, by hybridisation in an aqueous solution containing 6×SSC, 5× Denhardt's, 1% SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al (1989). Typically, constructs according to the invention comprise a promoter by the gene of interest, and a coding sequence encoding the desired reporter constructs, for example of GFP or luciferase. Vectors encoding GFP and luciferase are known in the art and available commercially. In a preferred embodiment, the 5′ regulatory region of a presenilin gene, especially human PS1 or PS2, or presenilin-dependent gamma-secretase gene is operatively joined to a reporter gene and cells are transformed with this recombinant construct. Such recombinant cells may then be used in high through-put assays for compounds which affect the expression of presenilin or presenilin-dependent gamma-secretase.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to, emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefor assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In viva Expression Technology (IVET) (Camilli). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

The invention, in certain embodiments, includes antibodies specifically recognising and binding to polypeptides.

Antibodies may be recovered from the serum of immunised animals. Monoclonal antibodies may be prepared from cells from immunised animals in the conventional manner.

The antibodies of the invention are useful for identifying cells expressing the genes being monitored.

Antibodies according to the invention may be whole antibodies of natural classes, such as IgE and IgM antibodies, but are preferably IgG antibodies. Moreover, the invention includes antibody fragments, such as Fab, F(ab′)₂, Fv and ScFv. Small fragments, such Fv and ScFv, possess advantageous properties for diagnostic and therapeutic applications on account of their small size and consequent superior tissue distribution.

The antibodies may comprise a label. Especially preferred are labels which allow the imaging of the antibody in neural cells in vivo. Such labels may be radioactive labels or radioopaque labels, such as metal particles, which are readily visualisable within tissues. Moreover, they may be fluorescent labels or other labels which are visualisable in tissues and which may be used for cell sorting.

For assays involving monitoring or detection of tolerised T cells for use in clinical applications, the assay will generally involve removal of a sample from a patient prior to the step of detecting a signal resulting from cleavage of the intracellular domain.

The invention additionally provides a method of screening for a candidate modulator of Notch IC protease activity and/or expression, the method comprising mixing in a buffer an appropriate amount of presenilin or presenilin-dependent gamma-secretase together with an appropriate amount of Notch, wherein Notch is suitably labelled with detection means for monitoring cleavage of Notch; and a sample of a candidate ligand; and monitoring any cleavage of Notch.

As used herein, the term “sample” refers to a collection of inorganic, organic or biochemical molecules which is either found in nature (e.g., in a biological- or other specimen) or in an artificially-constructed grouping, such as agents which may be found and/or mixed in a laboratory. The biological sample may refer to a whole organism, but more usually to a subset of its tissues, cells or component parts (e.g. body fluids, including but not limited to blood, mucus, saliva and urine).

Further methods of identifying presenilin or presenilin-dependent gamma-secretase modulators are described below. Presenilin or presenilin-dependent gamma-secretase modulators could be, for example, enzymes, co-receptors, ligands or stabilisers. The interaction of these compounds with presenilin or presenilin-dependent gamma-secretase will be indicative of a modulating function. Assays for the detection and/or analysis of such interactions are therefore included within the scope of the invention.

Soluble recombinant presenilin or presenilin-dependent gamma-secretase fusion proteins can be made, or the nucleotide sequence coding for presenilin or presenilin-dependent gamma-secretase amino acids (in particular functional domain amino acids) can be expressed, in suitable vectors (yeast-2-hybrid, baculovirus, and phage-display systems for instance) and used to identify proteins which interact with PS1 or PS2. Therapies can be designed to modulate these interactions and thus to modulate diseases of the immune system and other conditions associated with acquired or inherited abnormalities of the PS1 or PS2 genes or their gene products. The potential efficacy of these therapies can be tested by analyzing the affinity and function of these interactions after exposure to the therapeutic agent by standard pharmacokinetic measurements of affinity (Kd and Vmax etc) using synthetic peptides or recombinant proteins corresponding to functional domains of the PS1 gene, the PS2 gene or other presenilin or presenilin-dependent gamma-secretase homologues.

Another method for assaying the effect of any interactions involving functional domains such as the hydrophilic loop of the presenilin protein is to monitor changes in the intracellular trafficking and post-translational modification of the relevant genes by in situ hybridization, immunohistochemistry, Western blotting and metabolic pulse-chase labelling studies in the presence of, and in the absence of, the therapeutic agents. A further method is to monitor the effects of “downstream” events including (i) changes in the intracellular metabolism, trafficking and targeting of APP and its products; (ii) changes in second messenger events, e.g., cAMP intracellular Ca⁺⁺ protein kinase activities, etc.

Four domains have been identified as providing functional specificity to the presenilins. These functional domains are (1) the N-terminus (unique sequence in PS1 and PS2); (2) the TM6→7 loop (clustered mutations in the flanking conserved hydrophobic sequences and unique internal sequence); (3) the TM1, TM2 domains and TM1→2 linking sequence (concentration of several familial AD mutations) and (4) the C-terminus. To isolate proteins that interact with these functional domains, screening for presenilin binding proteins is carried out using GST-fusion constructs and synthetic peptides corresponding to these regions. For example, for PS2, GST-fusion peptides are made including sequences corresponding to amino acids 1 to 87 (N-terminus) or 272-390 (TM6→7 loop) or a synthetic peptide is made corresponding to amino acids 107 to 134 (TM1→2 link); for PS1, GST-fusion peptides are made including sequences corresponding to amino acids 1 to 81 (N-terminus) or 266 to 410 (TM6→7 loop) or a synthetic peptide is made corresponding to amino acids 101 to 131 (TM1→2 link). The following methods may be employed to isolate presenilin or presenilin-dependent gamma-secretase binding proteins:

(1) direct extraction by affinity chromatography using GST-fusion proteins and synthetic peptides;

(2) co-isolation of presenilins and bound proteins by immunoprecipitation;

(3) Biomolecular Interaction Assay (BIAcore) utilizing a GST-fusion capture system; and

(4) Two-Hybrid yeast systems.

GST-fusion proteins containing the N-terminus and TM6→7 loop sequences for PS1 and PS2 are used to probe human patient tissues and the isolated collection of proteins is separated by SDS-PAGE and microsequenced (Phizicky and Fields, 1995). To ensure that the band being sequenced contains only one protein species, the presenilin-fusion or presenilin-dependent gamma-secretase-fusion and binding proteins are separated by 2D gel electrophoresis prior to transfer and sequencing. For proteins with a blocked N-terminus, an additional HPLC purification and cleavage (CNBr and/or trypsin) of the particular binding protein is used to release peptide fragments. Further purification by HPLC and microsequencing by conventional methods provides internal sequence data on such blocked proteins.

The TM1→2 linking sequence is predicted to reside on the opposite side of the membrane to that of the N-terminal and TM6→7 loop and may be important in transmembrane communication. This is supported by the Tyr115His mutation which was observed in a pedigree with early onset familial AD (30-40 years) and by additional mutations in the TM1/2 helices which might be expected to destabilise the loop. The TM1→2 loop is relatively short (PS1: residues 101-131; PS2: residues 107-134) making this sequence more amenable to conventional peptide synthesis. The PS1 fragment (31-mer) has been synthesised containing an additional C-terminal cysteine residue. This peptide will be used to create an affinity substrate for affinity chromatography (Sulfo-link; Pierce) to isolate binding proteins for microsequencing. A peptide corresponding to the PS2 sequence is similarly synthesised and used to screen for distinct binding proteins.

An additional technique for the isolation of presenilins or presenilin-dependent gamma-secretases and their associated proteins is direct immunoprecipitation with antibodies. This procedure has been successfully used, for example, to isolate many of the synaptic vesicle associated proteins.

A useful method for the detection and isolation of binding proteins is the BIAcore system developed by Pharmacia Biosensor and described in the manufacturer's protocol (LKB Pharmacia, Sweden). This system uses an affinity purified anti-GST antibody to immobilise GST-fusion proteins onto a sensor chip. The sensor uses surface plasmon resonance which is an optical phenomenon that detects changes in refractive indices. A homogenate of a tissue of interest is passed over the immobilised fusion protein and protein-protein interactions are registered as changes in the refractive index. This system can be used to determine the kinetics of binding, to assess whether any observed binding is of physiological relevance.

The Two-Hybrid system takes advantage of transcriptional factors that are composed of two physically separable, functional domains (Fields and Stemglanz). The most commonly used is the yeast GAL4 transcriptional activator consisting of a DNA binding domain and a transcriptional activation domain. Two different cloning vectors are used to generate separate fusions of the GAL4 domains to genes encoding potential binding proteins. The fusion proteins are co-expressed, targeted to the nucleus and, if interactions occur, activation of a reporter gene (e.g. lacZ) produces a detectable phenotype. For example, the Clontech Matchmaker System-2 may be used to screen the Clontech cDNA GAL4 activation domain fusion library with presenilin GAL4 binding domain fusion clones (Clontech, Palo Alto, Calif.).

Small molecule-based therapies are particularly preferred because such molecules are more readily absorbed after oral administration and/or have fewer potential antigenic determinants than larger, protein-based pharmaceuticals. In light of the present disclosure, one of ordinary skill in the art will be able to develop drug screening methodologies which will be useful in the identification of candidate small molecule pharmaceuticals for the treatment of immune diseases. In particular, the skilled person will be able to screen large libraries of small molecules in order to identify those which bind to the normal and/or mutant PS1 or PS2 protein and which, therefore, are candidates for modifying the in vivo activity of the normal or mutant presenilin or presenilin-dependent gamma-secretase proteins. Furthermore, the skilled person will be able to identify small molecules which selectively or preferentially bind to a mutant form of a presenilin protein or presenilin-dependent gamma-secretase.

Methods for screening small molecule libraries for candidate protein-binding molecules are well known in the art and, in light of the present disclosure, may now be employed to identify compounds which bind to the normal or mutant forms of a presenilin or presenilin-dependent gamma-secretase.

Briefly, in one embodiment, either a normal or mutant PS1 or PS2 protein may be immobilised on a substrate such as a column or filter, and a solution including the test compound(s) is contacted with the presenilin protein under conditions which are permissive for binding. The substrate is then washed with a solution which substantially reflects physiological conditions to remove unbound or weakly bound small molecules. A second wash may then elute those compounds which strongly bound to the immobilised normal or mutant presenilin. Alternatively, the small molecule test compounds may be immobilised and a solution of normal or mutant PS1 or PS2 may be contacted with the column, filter or other substrate. The ability of the presenilin to bind to the small molecules may be determined as above or a labelled form of presenilin (e.g., radio-labelled or chemiluminescent) may be used to more rapidly assess binding to the substrate-immobilised compound(s).

In addition, as both PS1 and PS2 are believed to be membrane associated proteins, it may be preferred that the presenilin proteins be incorporated into lipid bilayers (e.g., liposomes) to promote their proper folding. Such presenilin-liposomes may be immobilised on substrates (either directly or by means of another element in the liposome membrane), passed over substrates with immobilised small molecules, or used in any of a variety of other well known binding assays for membrane proteins. In another series of embodiments, either normal or mutant, free or membrane-bound PS1 or PS2 may be mixed in a solution with the candidate compound(s) under conditions which are permissive for binding, and the presenilin may be immunoprecipitated. Small molecules which co-immunoprecipitate with a presenilin may then be identified. As will be obvious to one of ordinary skill in the art, there are numerous other methods of screening individual small molecules or large libraries of small molecules (e.g., phage display libraries) to identify compounds which bind to normal or mutant presenilins or presenilin-dependent gamma-secretase. All of these methods comprise the step of mixing normal or mutant presenilin or presenilin-dependent gamma-secretase with test compounds, allowing for binding (if any), and assaying for bound complexes.

Compounds which bind to normal or mutant or both forms of presenilins or presenilin-dependent gamma-secretase may have utility in treatments. Compounds which bind only to a normal presenilin or presenilin-dependent gamma-secretase may, for example, act as enhancers of its normal activity and thereby at least partially compensate for the lost or abnormal activity of mutant forms of the presenilin or presenilin-dependent gamma-secretase in patients suffering from immune diseases. Compounds which bind to both normal and mutant forms of a presenilin or presenilin-dependent gamma-secretase may have utility if they differentially affect the activities of the two forms so as to alleviate the overall departure from normal function.

Alternatively, blocking the activity of both normal and mutant forms of either PS1 or PS2 in heterozygotes may have less severe physiological and clinical consequences than the normal progress of the disease and, therefore, compounds which bind to and inhibit the activity of both normal and mutant forms of a presenilin may have utility. Preferably, however, compounds are identified which have a higher affinity of binding to mutant presenilin than to normal presenilin (e.g., 5-10 fold higher Kₐ) and which selectively or preferentially inhibit the activity of the mutant form. Such compounds may be identified by using any of the techniques described above and by then comparing the binding affinities of the candidate compound(s) for the normal and mutant forms of PS1 or PS2.

Once identified by the methods described above, the candidate compounds may then be produced in quantities sufficient for pharmaceutical administration or testing or may serve as “lead compounds” in the design and development of new pharmaceuticals. For example, as in well known in the art, sequential modification of small molecules (e.g., amino acid residue replacement with peptides; functional group replacement with peptide or non-peptide compounds) is a standard approach in the pharmaceutical industry for the development of new pharmaceuticals. Such development generally proceeds from a “lead compound” which is shown to have at least some of the activity (e.g., PS1 binding ability) of the desired pharmaceutical. In particular, when one or more compounds having at least some activity of interest (e.g., PS1 binding) are identified, structural comparison of the molecules can greatly inform the skilled practitioner by suggesting portions of the lead compounds which should be conserved and portions which may be varied in the design of new candidate compounds. Thus, the present invention also provides a means of identifying lead compounds which may be sequentially modified to produce new candidate compounds for use in the treatment of immune disease. These new compounds then may be tested both for presenilin-binding or presenilin-dependent gamma-secretase-binding (e.g., in the binding assays described above) and for therapeutic efficacy (e.g., in the animal models described herein). This procedure may be iterated until compounds having the desired therapeutic activity and/or efficacy are identified.

In another series of embodiments, the present invention provides assays for identifying small molecules or other compounds which are capable of inducing or inhibiting the expression of PS1, PS2 or other presenilin-related or presenilin-dependent gamma-secretase-related genes and proteins. The assays may be performed in vitro using non-transformed cells, immortalised cell lines, or recombinant cell lines. In addition, the assays may detect the presence of increased or decreased expression of PS1, PS2 or other presenilin-related or presenilin-dependent gamma-secretase— related genes or proteins on the basis of increased or decreased mRNA expression (using, e.g., the nucleic acid probes disclosed and enabled herein), increased or decreased levels of PS1, PS2 or other presenilin-related or presenilin-dependent gamma-secretase-related protein products (using, e.g., the anti-presenilin or presenilin-dependent gamma-secretase antibodies disclosed and enabled herein), or increased or decreased levels of expression of a reporter gene (e.g., β-galactosidase or luciferase) operatively joined to a presenilin or presenilin-dependent gamma-secretase 5′ regulatory region in a recombinant construct.

Thus, for example, one may culture cells known to express a particular presenilin or presenilin-dependent gamma-secretase and add to the culture medium one or more test compounds. After allowing a sufficient period of time (e.g., 6-72 hours) for the compound to induce or inhibit the expression of the presenilin or presenilin-dependent gamma-secretase, any change in levels of expression from an established baseline may be detected using any of the techniques described above and well known in the art. In particularly preferred embodiments, the cells are from an immortalised cell line such as a human glioblastoma cell line or a hybridoma-glioma cell line. Using the nucleic acid probes and/or antibodies disclosed and enabled herein, detection of changes in the expression of a presenilin or presenilin-dependent gamma-secretase, and thus identification of the compound as an inducer or repressor of presenilin or presenilin-dependent gamma-secretase expression, requires only routine experimentation.

In particularly preferred embodiments, a recombinant assay is employed in which a reporter gene such a β-galactosidase or luciferase is operably joined to the 5′ regulatory regions of a presenilin or presenilin-dependent gamma-secretase gene. Such regulatory regions may be easily isolated and cloned by one of ordinary skill in the art in light of the present disclosure of the coding regions of these genes. The reporter gene and regulatory regions are joined in-frame (or in each of the three possible reading frames) so that transcription and translation of the reporter gene may proceed under the control of the presenilin or presenilin-dependent gamma-secretase regulatory elements. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high through-put assay for the identification of inducers and repressors of the presenilin or presenilin-dependent gamma-secretase gene.

Compounds identified by this method will have potential utility in modifying the expression of the PS1, PS2 or other presenilin-related or presenilin-dependent gamma-secretase-related genes in vivo. These compounds may be further tested in the animal models disclosed and enabled herein to identify those compounds having the most potent in vivo effects. In addition, as described above with respect to small molecules having presenilin-binding or presenilin-dependent gamma-secretase-binding activity, these molecules may serve as “lead compounds” for the further development of pharmaceuticals by, for example, subjecting the compounds to sequential modifications, molecular modelling, and other routine procedures employed in rational drug design.

Candidate Compounds

The compound of the invention may be an organic compound or other chemical. In one preferred embodiment, the compound will be an amino acid sequence or a chemical derivative thereof, or a combination thereof. In another preferred embodiment, the compound will be a nucleotide sequence—which may be a sense sequence or an anti-sense sequence. The compound may also be an antibody.

Alternatively, the compound will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The compound may comprise at least one cyclic group. The cyclic group may be a polycyclic group, such as a non-fused polycyclic group. For some applications, the agent comprises at least the one of said cyclic groups linked to another hydrocarbyl group.

Polypeptides and Polynucleotides

Amino Acid Sequences

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

“Peptide” usually refers to a short amino acid sequence that is 10 to 40 amino acids long, preferably 10 to 35 amino acids.

The amino acid sequence may be prepared and isolated from a suitable source, or it may be made synthetically or it may be prepared by use of recombinant DNA techniques.

Nucleotide Sequences

As used herein, the term “nucleotide sequence” is synonymous with the term “polynucleotide”.

The nucleotide sequence may be DNA or RNA of genomic or synthetic or of recombinant origin. They may also be cloned by standard techniques. The nucleotide sequence may be double-stranded or single-stranded whether representing the sense or antisense strand or combinations thereof.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector. In general, primers will be produced by synthetic means, involving a step wise manufacture of the desired nucleic acid sequence one nucleotide at a time. Techniques for accomplishing this using automated techniques are readily available in the art.

“Polynucleotide” refers to a polymeric form of nucleotides of at least 10 bases in length and up to 1,000 bases or even more, either ribonucleotides or deoxyribonucleotides or a modified form of either type of nucleotide. The term includes single and double stranded forms of DNA.

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art.

For some applications, preferably, the nucleotide sequence is DNA. For some applications, preferably, the nucleotide sequence is prepared by use of recombinant DNA techniques (e.g. recombinant DNA). For some applications, preferably, the nucleotide sequence is cDNA. For some applications, preferably, the nucleotide sequence may be the same as the naturally occurring form.

The nucleotide sequence may comprise, for example, a protein-encoding domain, an antisense sequence or a functional motif such as a protein-binding domain and includes variants, derivatives, analogues and fragments thereof. The term also refers to polypeptides encoded by the nucleotide sequence.

Variants, Derivatives, Analogues, Homologues and Fragments

In addition to the specific amino acid sequences and nucleotide sequences mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be modified by addition, deletion, substitution modification replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| Symbol | 3-letter | Meaning | Codons |
|---|---|---|---|
| A | Ala | Alanine | GCT, GCC, GCA, GCG |
| B | Asp, Asn | Aspartic, | GAT, GAC, AAT, AAC |
| | | Asparagine | |
| C | Cys | Cysteine | TGT, TGC |
| D | Asp | Aspartic | GAT, GAC |
| E | Glu | Glutamic | GAA, GAG |
| F | Phe | Phenyl- | TTT, TTC |
| | | alanine | |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, |
| | | | CTA, CTG |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, CGG, |
| | | | AGA, AGG |
| S | Ser | Serine | TCT, TCC, TCA, TCG, |
| | | | AGT, AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, Gln | Glutamic, | GAA, GAG, CAA, CAG |
| | | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleodtide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Polynucleotide variants will preferably comprise codon optimised sequences. Codon optimisation is known in the art as a method of enhancing RNA stability and therefor gene expression. The redundancy of the genetic code means that several different codons may encode the same amino-acid. For example, Leucine, Arginine and Serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms. Preferably, at least part of the sequence is codon optimised. Even more preferably, the sequence is codon optimised in its entirety.

As used herein, the term “homology” can be equated with “identity”. A homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (University of Wisconsin, U.S.A.; Devereux). Examples of other software than can perform sequence comparisons include, but are not limited to, the BLAST package, FASTA (Atschul) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleotide sequences which are homologous to or variants of sequences of use in the present invention can be obtained in a number of ways, for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the polynucleotide or encoded polypeptide.

Immunotherapy

The modulators of the present invention including those identified by the assay method of the present invention may be used as therapeutic agents—i.e. in therapy applications.

The term “therapy” includes curative effects, alleviation effects, and prophylactic effects. The therapy may be on humans or animals.

Such modulators of the present invention may be used in immunotherapy, i.e. to treat disorders and/or conditions of the immune system. In particular, the compounds can be used in the treatment of T cell mediated diseases or disorders. A detailed description of the conditions affected by the Notch signalling pathway may be found in our WO98/20142, WO00/36089 and WO/00135990.

Diseased or infectious states that may be described as being mediated by T cells include, but are not limited to, any one or more of asthma, allergy, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple sclerosis, rheumatoid arthritis and diabetes. The present invention may also be used in organ transplantation or bone marrow transplantation. The present invention is also useful in treating immune disorders such as autoimmune disorders or graft rejection such as allograft rejection.

Examples of autoimmune disorders range from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail, organ-specific autoimmune diseases include multiple sclerosis, insulin dependent diabetes mellitus, several forms of anemia (aplastic, hemolytic), autoimmune hepatitis, thyroiditis, insulitis, iridocyclitis, skleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The present invention is also useful in cancer therapy, particularly in diseases involving the conversion of epithelial cells to cancer. The present invention is especially useful in relation to adenocarcinomas such as: small cell lung cancer, and cancer of the kidney, uterus, prostrate, bladder, ovary, colon and breast.

The present invention thus provides a method for enhancing the reactivity of a T cell toward a tumour cell.

In more detail, the T cells, APCs and/or tumour cells prepared by the method of the invention may be administered to a patient suffering from a malignancy, the malignancy typically comprising cancerous cells that express a Notch ligand. The presence of cancerous cells that express, in particular over-express, a Notch ligand may be determined by, for example, testing using the methods described above a sample of cancerous tissue obtained from the patient.

Generally, the patient will be the same patient from whom the treated T cells, APCs and/or tumour cells originated. Examples of malignancies that may be treated include cancer of the breast, cervix, colon, rectum, endometrium, kidney, lung, ovary, pancreas, prostate gland, skin, stomach, bladder, CNS, oesophagus, head-or-neck, liver, testis, thymus or thyroid. Malignancies of blood cells, bone marrow cells, B-lymphocytes, T-lymphocytes, lymphocytic progenitors or myeloid cell progenitors may also be treated.

The tumour may be a solid tumour or a non-solid tumour and may be a primary tumour or a disseminated metastatic (secondary) tumour. Non-solid tumours include myeloma; leukaemia (acute or chronic, lymphocytic or myelocytic) such as acute myeloblastic, acute promyelocytic, acute myelomonocytic, acute monocytic, erythroleukaemia; and lymphomas such as Hodgkin's, non-Hodgkin's and Burkitt's. Solid tumours include carcinoma, colon carcinoma, small cell lung carcinoma, non-small cell lung carcinoma, adenocarcinoma, melanoma, basal or squamous cell carcinoma, mesothelioma, adenocarcinoma, neuroblastoma, glioma, astrocytoma, medulloblastoma, retinoblastoma, sarcoma, osteosarcoma, rhabdomyosarcoma, fibrosarcoma, osteogenic sarcoma, hepatoma, and seminoma.

The tumour may be one which presents intracellular or membrane-bound antigens including tumour-specific antigens (for example virally encoded antigens, neo-antigens such as MUC1, antibody idiotypes); antigens which are overexpressed on the surface of tumour cells; oncofoetal antigens including cancer-testis (CT) antigens; or differentiation-antigens (such as tyrosinase and melanocyte antigens). The patient may have an ongoing immune response, such as a Th1 or Th2-type immune response, to antigens on the tumour and may have detectable cytotoxic T cell (CTL) activity, NK cell activity and/or antibody responses against the tumour as determined by, for example, in vitro assays.

Alternatively, the APCs and/or lymphocytes of the present invention can be used to efficiently transfer infectious tolerance to a chosen antigen or antigens when transferred into a patient for the treatment of a disease characterised by inappropriate lymphocyte activity, such as Th1 or Th2 cell activity. The APCs and/or lymphocytes may thus be used to treat an ongoing immune response (such as an allergic condition or an autoimmune disease) or may be used to generate tolerance in an immunologically lymphocytes cells of the present invention may be used in therapeutic methods for both treating and preventing diseases characterised by inappropriate lymphocyte activity in animals and humans. The APCs and/or lymphocytes may be used to confer tolerance to a single antigen or to multiple antigens. Typically, APCs and/or lymphocytes are obtained from the patient or donor and primed as described above before being returned to the patient (ex vivo therapy).

The present invention may also be employed to produce a lymphocyte or APC having tolerance to an allergen or antigen.

Antigens and Allergens

An antigen may be any substance that can be recognised generally as foreign, by the immune system, and is generally recognised by an antigen receptor. Preferably the antigen used in the present invention is an immunogen. An allergic response occurs when the host is re-exposed to an antigen that it has encountered previously.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNγ but high IL-4, IL-5, IL-10. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen or allergen used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component. In particular, it is preferred to use antigens known to be associated with auto-immune diseases such as myelin basic protein (associated with multiple sclerosis), collagen (associated with rheumatoid arthritis), and insulin (diabetes), or antigens associated with rejection of non-self tissue such as MHC antigens. Where primed the APCs and/or T cells of the present invention are to be used in tissue transplantation procedures, antigens will be obtained from the tissue donor.

The antigen or allergen moiety may be, for example, a synthetic MHC-peptide complex i.e. a fragment of the MHC molecule bearing the antigen groove bearing an element of the antigen. Such complexes have been described in Altman et al., 1996.

Tumor-associated antigens which may be used include, for example:

beta chain of human chorionic gonadotropin (hCG beta) antigen, carcinoembryonic antigen, EGFRvIII antigen, Globo H antigen, GM2 antigen, GP100 antigen, HER2/neu antigen, KSA antigen, Le (y) antigen, MUC1 antigen, the MAGE family of antigens (such as MAGE 1 antigen, MAGE 2 antigen, MAGE-4A antigen), MUC2 antigen, MUC3 antigen, MUC4 antigen, MUC5AC antigen, MUC5B antigen, MUC7 antigen, PSA antigen, PSCA antigen, PSMA antigen,

Thompson-Friedenreich antigen (TF), Tn antigen, sTn antigen, TRP 1 antigen, TRP 2 antigen, tumor-specific immunoglobulin variable region and tyrosinase antigen, Wilms tumour gene (WT1), KH-1 antigen, p53, RAS, heat shock proteins (HSP) such as HSP70 and HSP10.

Active fragments of such antigens and antigenic determinants having similar or equivalent activity may also be used. Antigens may be administered, for example, as discrete proteins/polypeptides or associated with whole or disrupted cells or membranes, or by administering poylnucleotides coding for such antigens or antigenic determinants so that the antigen or antigenic determinant is expressed in the subject.

Alternatively or in addition autologous or heterologous tumour cells or derivatives may be used. For example, whole cell antigen preparations may be employed such as tumour cells and tumour/APC hybrid cells.

Preparation of Primed APCs and Lymphocytes

Preparation of Primed APCs ex Vivo in the Absence of Lymphocytes

APCs as described above are cultured in a suitable culture medium such as DMEM or other defined media, optionally in the presence of fetal calf serum. Cytokines, if present, are typically added at up to 1000 U/ml. Optimum concentrations may be determined by titration. One or more substances capable of modulating presenilin and, optionally, one or more substances capable of up-regulating or down-regulating the Notch signalling pathway are then typically added to the culture medium together with the antigen of interest. The antigen may be added before, after or at substantially the same time as the substance(s). Cells are typically incubated with the substance(s) and antigen for at least one hour, preferably at least 3 hours, suitably at least 24 to 72 hours at 37° C. If required, a small aliquot of cells may be tested for modulated target gene expression as described above. Alternatively, cell activity may be measured by the inhibition of T cell proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction.

The resulting APCs that show increased levels of a Notch signalling are now ready for use.

Preparation of Regulatory T Cells (and B Cells) Ex Vivo

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s) capable of modulating presenilin to that used with the APC, then the T cell will not be brought into contact with the different substance(s) used in the APC. Alternatively, the T cell may be incubated with a first substance (or set of substances) to modulate presenilin or presenilin-dependent gamma-secretase and, optionally, Notch signalling, washed, resuspended and then incubated with the primed APC in the absence of both the substance(s) used to modulate the APC and the substance(s) used to modulate the T cell. Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Incubations will typically be for at least 1 hour, preferably at least 3 or 6 hours, suitably about 48 to 72 hours in suitable culture medium at 37° C. The progress of presenilin or presenilin-dependent gamma-secretase modulation may be determined for a small aliquot of cells using the methods described above. T cells transfected with a nucleic acid construct directing the expression of, for example Delta, may be used as a control. Induction of immunotolerance may be determined by subsequently challenging T cells with antigen and measuring IL-2 production compared with control cells not exposed to APCs.

Primed T cells or B cells may also be used to induce immunotolerance in other T cells or B cells in the absence of APCs using similar culture techniques and incubation times.

Pharmaceutical Compositions

The present invention provides a pharmaceutical composition comprising administering a therapeutically effective amount of the compound identified by the method of the present invention and a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the compound is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Vaccine Compositions

Vaccine compositions and preparations made in accordance with the present invention may be used to protect or treat a mammal susceptible to, or suffering from disease, by means of administering said vaccine via a mucosal route, such as the oral/bucal/intestinal/vaginal/rectal or nasal route. Such administration may be in a droplet, spray, or dry powdered form. Nebulised or aerosolised vaccine formulations may also be used where appropriate.

Enteric formulations such as gastro resistant capsules and granules for oral administration, suppositories for rectal or vaginal administration may also be used. The present invention may also be used to enhance the immunogenicity of antigens applied to the skin, for example by intradermal, transdermal or transcutaneous delivery. In addition, the adjuvants of the present invention may be parentally delivered, for example by intramuscular or subcutaneous administration.

Depending on the route of administration, a variety of administration devices may be used. For example, for intranasal administration a spray device such as the commercially available Accuspray (Becton Dickinson) may be used.

Preferred spray devices for intranasal use are devices for which the performance of the device is not dependent upon the pressure applied by the user. These devices are known as pressure threshold devices. Liquid is released from the nozzle only when a threshold pressure is attained. These devices make it easier to achieve a spray with a regular droplet size. Pressure threshold devices suitable for use with the present invention are known in the art and are described for example in WO 91/13281 and EP 311 863 B. Such devices are commercially available from Pfeiffer GmbH.

For certain vaccine formulations, other vaccine components may be included in the formulation. For example the adjuvant formulations of the present invention may also comprise a bile acid or derivative of cholic acid. Suitably the derivative of cholic acid is a salt thereof, for example a sodium salt thereof. Examples of bile acids include cholic acid itself, deoxycholic acid, chenodeoxy colic acid, lithocholic acid, taurodeoxycholate ursodeoxycholic acid, hyodeoxycholic acid and derivatives like glyco-, tauro-, amidopropyl-1-propanesulfonic- and amidopropyl-2-hydroxy-1-propanesulfonic-derivatives of the above bile acids, or N,N-bis (3DGluconoamidopropyl) deoxycholamide.

Suitably, the adjuvant formulation of the present invention may be in the form of an aqueous solution or a suspension of non-vesicular forms. Such formulations are convenient to manufacture, and also to sterilise (for example by terminal filtration through a 450 or 220 nm pore membrane).

Suitably, the route of administration to said host is via the skin, intramuscular or via a mucosal surface such as the nasal mucosa. When the admixture is administered via the nasal mucosa, the admixture may for example be administered as a spray. The methods to enhance an immune response may be either a priming or boosting dose of the vaccine.

The term “adjuvant” as used herein includes an agent having the ability to enhance the immune response of a vertebrate subject's immune system to an antigen or antigenic determinant.

The term “immune response” includes any response to an antigen or antigenic determinant by the immune system of a subject. Immune responses include for example humoral immune responses (e.g. production of antigen-specific antibodies) and cell-mediated immune responses (e.g. lymphocyte proliferation, cytokine secretion and cytotoxic activity).

The term “cell-mediated immune response” includes the immunological defence provided by lymphocytes, such as the defence provided by T cell lymphocytes when they come into close proximity with their victim cells.

When “lymphocyte proliferation” is measured, the ability of lymphocytes to proliferate in response to specific antigen may be measured. Lymphocyte proliferation includes B cell, T-helper cell or CTL cell proliferation.

Compositions of the present invention may be used to formulate vaccines containing antigens derived from a wide variety of sources. For example, antigens may include human, bacterial, or viral nucleic acid, pathogen derived antigen or antigenic preparations, host-derived antigens, including GnRH and IgE peptides, recombinantly produced protein or peptides, and chimeric fusion proteins.

Preferably the vaccine formulations of the present invention contain an antigen or antigenic composition capable of eliciting an immune response against a human pathogen. The antigen or antigens may, for example, be peptides/proteins, polysaccharides and lipids and may be derived from pathogens such as viruses, bacteria and parasites/fungi as follows:

Viral Antigens

Viral antigens or antigenic determinants may be derived, for example, from:

Cytomegalovirus (especially Human, such as gB or derivatives thereof); Epstein Barr virus (such as gp350); flaviviruses (e.g. Yellow Fever Virus, Dengue Virus, Tick-borne encephalitis virus, Japanese Encephalitis Virus); hepatitis virus such as hepatitis B virus (for example Hepatitis B Surface antigen such as the PreS1, PreS2 S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992, 10, 915-920) or purified or recombinant proteins thereof, such as NP, NA, HA, or M proteins); measles virus; mumps virus; parainfluenza virus; Respiratory Syncytial virus (such as F and G proteins); rotavirus (including live attenuated viruses); Varicella Zoster Virus (such as gpI, II and IE63); and Human Papilloma Virus (HPV) considered to be responsible for genital warts, (HPV 6 or HPV 11 and others), and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Bacterial Antigens

Bacterial antigens or antigenic determinants may be derived, for example, from:

Bacillus spp., including B. anthracis (eg botulinum toxin); Bordetella spp, including B. pertussis (for example pertactin, pertussis toxin, filamenteous hemagglutinin, adenylate cyclase, fimbriae); Borrelia spp., including B. burgdorferi (eg OspA, OspC, DbpA, DbpB), B. garinii (eg OspA, OspC, DbpA, DbpB), B. afzelii (eg OspA, OspC, DbpA, DbpB), B. andersonii (eg OspA, OspC, DbpA, DbpB), B. hermsii; Campylobacter spp, including C. jejuni (for example toxins, adhesins and invasins) and C. coli; Chlamydia spp., including C. trachomatis (eg MOMP, heparin-binding proteins), C. pneumonie (eg MOMP, heparin-binding proteins), C. psittaci; Clostridium spp., including C. tetani (such as tetanus toxin), C. botulinum (for example botulinum toxin), C. difficile (eg clostridium toxins A or B); Corynebacterium spp., including C. diphtheriae (eg diphtheria toxin); Ehrlichia spp., including E. equi and the agent of the Human Granulocytic Ehrlichiosis; Rickettsia spp, including R. rickettsii;

Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (eg PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa;

Legionella spp, including L. pneumophila; Leptospira spp., including L. interrogans; Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example WO97/41731)); Mycobacterium spp., including M. tuberculosis (for example ESAT6, Antigen 85A, -B or -C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (eg capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (see for example WO 90/06951; WO 99/03884); Treponema spp., including T. pallidum (eg the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasite/Fungal Antigens

Parasitic/fungal antigens or antigenic determinants may be derived, for example, from:

Babesia spp., including B. microti; Candida spp., including C. albicans; Cryptococcus spp., including C. neoformans; Entamoeba spp., including E. histolytica; Giardia spp., including; G. lamblia; Leshmania spp., including L. major; Plasmodium faciparum (MSP1, AMA1, MSP3, EBA, GLURP, RAP1, RAP2, Sequestrin, PFEMPI, Pf332, LSA1, LSA3, STARP, SALSA, PFEXPI, Pfs25, Pfs28, PFS27/25, Pfs16, Pfs48/45, Pfs230 and their analogues in Plasmodium spp.);

Pneumocystis spp., including P. carinii; Schisostoma spp., including S. mansoni; Trichomonas spp., including T. vaginalis; Toxoplasma spp., including T. gondii (for example SAG2, SAG3, Tg34); Trypanosoma spp., including T. cruzi.

It will be appreciated that in accordance with this aspect of the present invention antigens and antigenic determinants may be used in many different forms. For example, antigens or antigenic determinants may be present as isolated proteins or peptides (for example in so-called “subunit vaccines”) or, for example, as cell-associated or virus-associated antigens or antigenic determinants (for example in either live or killed pathogen strains). Live pathogens will preferably be attenuated in known manner. Alternatively, antigens or antigenic determinants may be generated in situ in the subject by use of a polynucleotide coding for an antigen or antigenic determinant (as in so-called “DNA vaccination”, although it will be appreciated that the polynucleotides which may be used with this approach are not limited to DNA, and may also include RNA and modified polynucleotides as discussed above).

The amount of protein in a vaccine dose is selected as an amount which induces an immunoprotective response without significant, adverse side effects in typical recipients. Such amount will vary depending upon which specific immunogen is employed and how it is presented. Typically, it is expected that each dose will comprise 1-1000 μg of protein, preferably 1-500 μg, preferably 1-100 μg, most preferably 1 to 50 μg. After an initial vaccination, subjects may receive one or several booster immunisations suitably spaced.

The vaccines of the present invention may also be administered via the oral route. In such cases the pharmaceutically acceptible excipient may also include alkaline buffers, or enteric capsules or microgranules. The vaccines of the present invention may also be administered by the vaginal route. In such cases, the pharmaceutically acceptable excipients may also include emulsifiers, polymers such as CARBOPOL, and other known stablilisers of vaginal creams and suppositories. The vaccines of the present invention may also be administered by the rectal route. In such cases the excipients may also include waxes and polymers known in the art for forming rectal suppositories.

The formulations of the present invention may be used for both prophylactic and therapeutic purposes. Accordingly, the present invention provides for a method of treating a mammal susceptible to or suffering from an infectious disease or allergy, or auto-immune disease. In a further aspect of the present invention there is provided an adjuvant combination and a vaccine as herein described for use in medicine. Vaccine preparation is generally described in New Trends and Developments in Vaccines, edited by Voller et al., University Park Press, Baltimore, Md., U.S.A. 1978.

It will be appreciated that the adjuvants of the present invention may further be combined with other adjuvants including, for example: Cholera toxin and its B subunit; E. Coli heat labile enterotoxin LT, its B subunit LTB and detoxified versions thereof such as mLT; immunologically active saponin fractions e.g. Quil A derived from the bark of the South American tree Quillaja Saponaria Molina and derivatives thereof (for example QS21, as described in U.S. Pat. No. 5,057,540); the oligonucleotide adjuvant system CpG (as described in WO 96/02555), especially 5′TCG TCG TTT TGT CGT TTT GTC GTT3 (SEQ ID NO. 1); and Monophosphoryl Lipid A and its non-toxic derivative 3-O-deacylated monophosphoryl lipid A (3D-MPL, as described in GB 2,220,211).

The present invention provides an increased magnitude and/or increased duration of immune response. Preferably the invention provides an increased protective immune response.

The present invention also contemplates generating selective Th1 or Th2 immunity. In general, T cells can act in different subpopulations that show different effector functions. T cell responses can be pro-inflammatory T helper 1 type (Th1) characterized by the secretion of interferon gamma (IFN-gamma.) and interleukin 2 (IL-2). Th1 cells are the helper cells for the cellular defence but provide little help for antibody secretion. The other class of T cell responses is generally anti-inflammatory, and is mediated by Th2 cells that produce IL-4, IL-5 and IL-10, but little or no IL-2 or IFN-gamma. Th2 cells are the helper cells for antibody production. CD4+ and CD8+cells both occur in these subpopulations: Th1/Th2:CD4, Tc1/Tc2:CD8.

For each type of pathogen/infection there may be an “appropriate” (and different) type of T cell response (e.g., Th1 vs. Th2, CD4+vs. CD8+) that combats the infectious agent but does not cause excessive tissue damage in the subject. It may be detrimental to the subject if an “inappropriate” type of T cell response is induced (Th1 instead of Th2, or vice versa). Generally, one would want to induce the Th1 response to clear an intracellular pathogen such as a virus or intracellular bacterium and a Th2 response to clear an extracellular pathogen.

It will be appreciated that the present invention may be used in both prophylactic and therapeutic vaccines. For example, prophylactic vaccines may be used to provide protective immunity to provide protection against establishment of infection. Therapeutic vaccines may be used, for example, after an infection has become established in order to increase the immune response against the infection. Suitably, therapeutic vaccines may be used to combat chronic infections which may for example be bacterial infections (such as tuberculosis), parasitic infections or viral infections (such as HPV, HCV, HBV or HIV infections).

In one embodiment the modulator/inhibitor of Notch signalling increases cytotoxic (CD8+) T cell responses to antigen.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration.

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular, intradermal, intra-articular, intrathecal, intra-peritoneal or subcutaneous route, or via the alimentary tract (for example, via the Peyers patches). Administration may also be by use of implants, e.g. subcutaneous implants as described in WO99/44583 (Applied Vaccine Technologies Corp).

Cells and pharmaceutical comprising cells of the invention are typically administered to the patient by intramuscular, intraperitoneal or intravenous injection, or by direct injection into the lymph nodes of the patient, preferably by direct injection into the lymph nodes. Typically from 10⁴ to 10⁸ treated cells, preferably from 10⁵ to 10⁷ cells, more preferably about 10⁶ cells are administered to the patient.

The routes of administration and dosages described are intended only as a guide since a skilled practitioner will be able to determine readily the optimum route of administration and dosage for any particular patient depending on, for example, the age, weight and condition of the patient. Preferably the pharmaceutical compositions are in unit dosage form. The present invention includes both human and veterinary applications.

By “simultaneously” is meant that the modulator of the Notch signalling pathway and the pathogen antigen, antigenic determinant or the polynucleotide coding for the pathogen antigen or antigenic determinant are administered at substantially the same time, and preferably together in the same formulation.

By “contemporaneously” it is meant that the modulator of the Notch signalling pathway and the pathogen antigen, antigenic determinant or the polynucleotide coding for the pathogen antigen or antigenic determinant are administered closely in time, e.g., the the pathogen antigen, antigenic determinant or the polynucleotide coding for the pathogen antigen or antigenic determinant is administered within from about one minute to within about one day before or after the modulator of the Notch signalling pathway is administered. Any contemporaneous time is useful. However, it will often be the case that when not administered simultaneously, the modulator of the Notch signalling pathway and the pathogen antigen, antigenic determinant or the polynucleotide coding for the pathogen antigen or antigenic determinant will be administered within about one minute to within about eight hours, and preferably within less than about one to about four hours. When administered contemporaneously, the modulator of the Notch signalling pathway and the pathogen antigen, antigenic determinant or the polynucleotide coding for the pathogen antigen or antigenic determinant are preferably administered at the same site on the animal.

The term “same site” includes the exact location, but can be within about 0.5 to about 15 centimeters, preferably from within about 0.5 to about 5 centimeters.

The term “separately” as used herein means that the modulator of the Notch signalling pathway and the pathogen antigen, antigenic determinant or the polynucleotide coding for the pathogen antigen or antigenic determinant are administered at an interval, for example at an interval of about a day to several weeks or months. The active agents may be administered in either order.

Likewise, the modulator of the Notch signalling pathway may be administered more frequently than the pathogen antigen, antigenic determinant or the polynucleotide coding for the pathogen antigen or antigenic determinant or vice versa.

The term “sequentially” as used herein means that the modulator of the Notch signalling pathway and the pathogen antigen, antigenic determinant or the polynucleotide coding for the pathogen antigen or antigenic determinant are administered in sequence, for example at an interval or intervals of minutes, hours, days or weeks. If appropriate the active agents may be administered in a regular repeating cycle.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

Various features and embodiments of the invention will now be described with reference to the following non-limiting Examples.

### EXAMPLES

### Example 1

### Effect of MW167 on Notch Signalling

C2C12 cells were transfected with plasmid pLOR3 (mHES1-Luc reporter construct) using Effectene transfection reagent (Qiagen) for 24 hours.

A 96-well tissue culture plate was coated overnight at 4° C. with 100 μl of anti-V5 monoclonal antibody (Invitrogen) diluted to 1 μg/ml in PBS. The following day the plate was washed with PBS and 100 μl of purified mDelta1-V5-His fusion protein (BD34) diluted to 2 μg/ml in PBS was added for 2 hours at room temperature. The plate was washed with PBS before adding the transfected C2C12 cells.

The transfected C2C12 cells were trypsinised, resuspended at 4.0×10⁵ cells/ml and plated out at 100 μl/well. The γ-secretase inhibitor, MW167 (Calbiochem γ-secretase inhibitor II, Cat. No. 565755), was dissolved in DMSO at 10 mM and added to duplicate wells at a final concentration of 100 μM. Control wells were set up with an equivalent dilution of DMSO alone. The plate was placed in a CO₂ incubator at 37° C. for 24 hours.

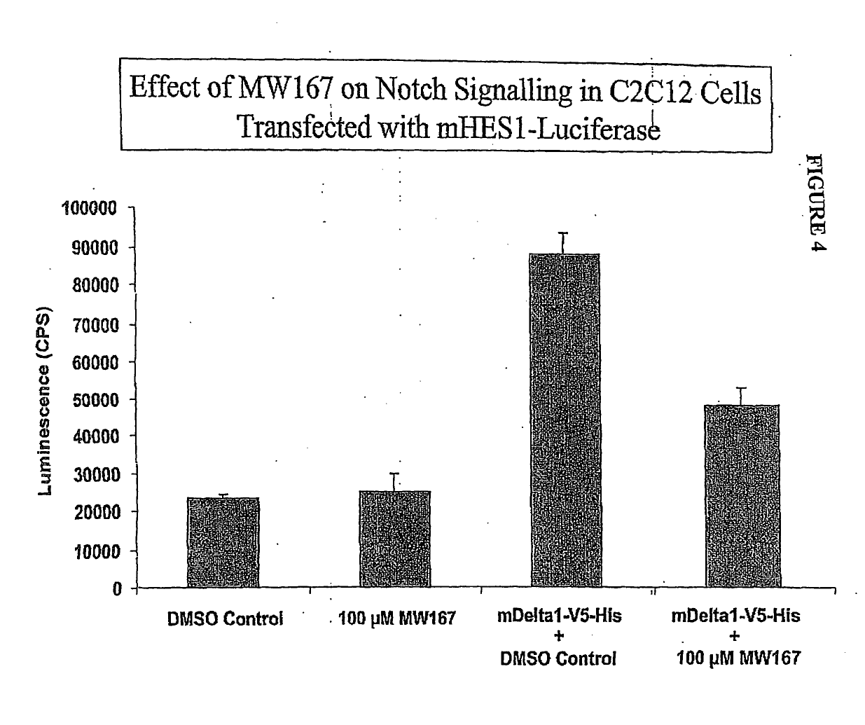

The supernatant was removed from all the wells and 100 μl of PBS was added followed by 100 μl of SteadyGlo luciferase assay regent (Promega). The plate was left for 5 minutes at room temperature and then 200 μl was removed and placed into a white 96-well OptiPlate (Packard) tissue culture plate and the luminescence read in a TopCount (Packard) counter. The results are shown in FIG. 4.

### Example 2

### hDelta1-IG4Fc Fusion Protein

A fusion protein comprising the extracellular domain of human Delta1 fused to the Fc domain of human IgG4 (“hDelta1-IgG4Fc”) was prepared by inserting a nucleotide sequence coding for the extracellular domain of human Delta1 (see, e.g. Genbank Accession No AF003522) into the expression vector pCONγ (Lonza Biologics, Slough, UK) and expressing the resulting construct in CHO cells. The amino acid sequence (SEQ ID NO:2) of the

MGSRCALALAVLSALLCQVWSSGVFELKLQEFVNKKGLLGNRNCCRGGAGPPPCA
CRTFFRVCLKHYQASVSPEPPCTYGSAVTPVLGVDSFSLPDGGGADSAFSNPIRFPFG
FTWPGTFSLIIEALHTDSPDDLATENPERLISRLATQRHLTVGEEWSQDLHSSGRTDL
KYSYRFVCDEHYYGEGCSVFCRPRDDAFGHFTCGERGEKVCNPGWKGPYCTEPICL
PGCDEQHGFCDKPGECKCRVGWQGRYCDECIRYPGCLHGTCQQPWQCNCQEGWG
GLFCNQDLNYCTHHKPCKNGATCTNTGQGSYTCSCRPGYTGATCELGDECDPSPC
KNGGSCTDLENSYSCTCPPGFYGKICELSAMTCADGPCFNGGRCSDSPDGGYSCRCP
VGYSGFNCEKKIDYCSSSPCSNGAKCVDLGDAYLCRCQAGFSGRHCDDNVDDCASS
PCANGGTCRDGVNDFSCTCPPGYTGRNCSAPVSRCEHAPCHNGATCHERGHGYVCE
CARGYGGPNCQFLLPELPPGPAVVDLTEKLEASTKGPSVFPLAYCSRSTSESTAALGC
LVKDYFPEPVTVSWNSGALTSGVHTFPAVLOSSGLYSLSSVVTVPSSSLGTKTYTCN
VDHKPSNTKVDKRVESKYGPPCPSCPAPEFLGGPSVFLFPPKPKDTLMISRTPEVTCV
VVDVSOEDPEVQFNWYVDGVEVHNAKTKPREEQFNSTYRVVSVLTVLHQDWLNG
KEYKCKVSNKGLPSSIEKTISKAKGOPREPOVYTLPPSQEEMTKNOVSLTCLVKGFY
PSDIAVEWESNGQPENNYKTTPPVLDSDGSFFLYSRLTVDKSRWOEGNVFSCSVMH
EALHNHYTOKSLSLSLGK

Wherein the first underlined sequence is the signal peptide (cleaved from the mature protein) and the second underlined sequence is the IgG4 Fc sequence.

### Example 3

### Modulation of Cytokine Production by γ-Secretase Inhibitor in Human CD4+ T Cells

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 40 minutes at 400 g. PBMC were recovered from the interface and washed 3 times before use for CD4+ T cell purification.

Human CD4+ T cells were isolated by positive selection using anti-CD4 microbeads from Miltenyi Biotech according to the manufacturer's instructions.

The CD4+ T cells were incubated in triplicates in a 96-well-plate (flat bottom) at 10⁵ CD4/well/200 ml in RPMI medium containing 10% FCS, glutamine, penicillin, streptomycin and b₂-mercaptoethanol.

Cytokine production was induced by stimulating the cells with anti-CD3/CD28 T cell expander beads from Dyna1 at a 1:1 ratio (bead/cell). Dyna1 beads coated with hDelta1-IgG4Fc fusion protein or control beads were added in some of the wells at a 5:1 ratio (beads/cell) and the γ-secretase inhibitor MW 167 (Calbiochem γ-secretase inhibitor II, Cat. No. 565755) was added variously (in DMSO) to final concentrations of 0, 0.4 mM, 2 mM and 10 mM.

The supernatants were removed after 3 days of incubation at 37° C./5% CO₂/humidified atmosphere and cytokine production was evaluated by ELISA using Pharmingen kits OptEIA Set human IL10 (catalog No. 555157), OptEIA Set human IL-5 (catalog No. 555202) for IL-10 and IL-5 respectively according to the manufacturer's instructions.

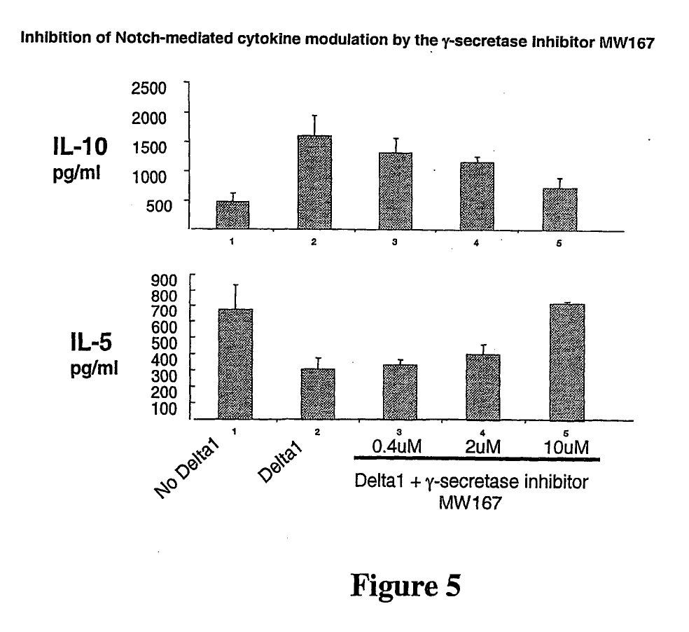

Results are shown in FIG. 5 from which it can be seen that the γ-secretase inhibitor substantially reversed a Delta-mediated increase in IL-10 expression and also substantially reversed a Delta-mediated reduction in IL-5 expression.

### Example 4

### Effect of γ-Secretase Inhibitor on Delta-Mediated Activation of Notch Signalling in Jurkat-N2 Cells

A) Construction of Luciferase Reporter Plasmid 10×CBF1-Luc (pLOR91)

An adenovirus major late promoter TATA-box motif with BglII and HindIII cohesive ends was generated as follows:

| | |
|---|---|
| BglII HindIII | |
| GATCTGGGGGGCTATAAAAGGGGGTA | (SEQ ID NO:3) |
| ACCCCCCGATATTTTCCCCCATTCGA | (SEQ ID NO:4) |

This was cloned into plasmid pGL3-Basic (Promega) between the BgiII and HindIII sites to generate plasmid pGL3-AdTATA.

A TP1 promoter sequence (TP1; equivalent to 2 CBF1 repeats) with BamH1 and BglII cohesive ends was generated as follows:

| | |
|---|---|
| BamH1 BglII | |
| 5′ GATCCCGACTCGTGGGAAAATGGGCGGAAGGGCACCGTGGGAAAATAGTA 3′ | (SEQ ID NO:5) |
| 3′ GGCTGAGCACCCTTTTACCCGCCTTCCCGTGGCACCCTTTTATCATCTAG 5′ | (SEQ ID NO:6) |

This sequence was pentamerised by repeated insertion into a BglII site and the resulting TP1 pentamer (equivalent to 10 CBF 1 repeats) was inserted into pGL3-AdTATA at the BglII site to generate plasmid pLOR91.

B) Generation of Notch2 Vector (pLOR92)

A cDNA clone spanning the complete coding sequence of the human Notch2 gene (see, e.g. GenBank Accession No AF315356) was constructed as follows. A 3′ cDNA fragment encoding the entire intracellular domain and a portion of the extracellular domain was isolated from a human placental cDNA library (OriGene Technologies Ltd., USA) using a PCR-based screening strategy. The remaining 5′ coding sequence was isolated using a RACE (Rapid Amplification of cDNA Ends) strategy and ligated onto the existing 3′ fragment using a unique restriction site common to both fragments (Cla I). The resulting full-length cDNA was then cloned into the mammalian expression vector pcDNA3.1-V5-HisA (Invitrogen) without a stop codon to generate plasmid pLOR92. When expressed in mammalian cells, pLOR92 thus expresses the full-length human Notch2 protein with V5 and His tags at the 3′ end of the intracellular domain.

C) Reporter Assay using Jurkat Cell Line

As Jurkat cells cannot be cloned by simple limiting dilution a methylcellulose-containing medium (ClonaCell™ TCS) was used with these cells.

Jurkat E6.1 cells (lymphoblast cell line; ATCC No TIB-152) were cloned using ClonaCell™ Transfected Cell Selection (TCS) medium (StemCell Technologies, Vancouver, Canada and Meylan, France) according to the manufacturer's guidelines.

Plasmid pLOR92 (prepared as described above) was electroporated into the Jurkat E6.1 cells with a Biorad Gene Pulser II electroporator as follows:

Actively dividing cells were spun down and resuspended in ice-cold RPMI medium containing 10% heat-inactivated FCS plus glutamine plus penicillin/streptomycin (complete RPMI) at 2.0×10⁷ cells per ml. After 10 min on ice, 0.5 ml of cells (ie 1×10⁷ cells) was placed into a pre-cooled 4 mm electroporation cuvette containing 20 μg of plasmid DNA (Endo-free Maxiprep DNA dissolved in sterile water). The cells were electroporated at 300 v and 950 μF and then quickly removed into 0.5 ml of warmed complete RPMI medium in an Eppendorf tube. The cells were spun for at 3000 rpm for 1 min in a microfuige and placed at 37° C. for 15 min to recover from being electroporated. The supernatant was then removed and the cells were plated out into a well of a 6-well dish in 4 ml of complete RPMI and left at 37° C. for 48 h to allow for expression of the antibiotic resistance marker.

After 48 h the cells were spun down and resupended into 10 ml fresh complete RPMI. This was then divided into 10×15 ml Falcon tubes and 8 ml of pre-warmed ClonaCell-TCS medium was added followed by 1 ml of a 10×final concentration of the antibiotic being used for selection. For G418 selection the final concentration of G418 was 1 mg/ml so a 10 mg/ml solution in RPMI was prepared and 1 ml of this was added to each tube. The tubes were mixed well by inversion and allowed to settle for 15 min at room temperature before being plated out into 10 cm tissue culture dishes. These were then placed in a CO₂ incubator for 14 days when that were examined for visible colonies.

Macroscopically visible colonies were picked off the plates and these colonies were expanded through 96-well plates to 24-well plates to T25 flasks—in complete RPMI containing 1 mg/ml G418.

The resulting clones were each transiently transfected with pLOR91 using Lipofectamine 2000 reagent (according to manufacturer's protocol) and then plated out onto a 96-well plate containing plate-bound immobilised hDelta1-IgG4Fc (prepared as described below). A well-performing clone (#24) was selected and used for further study.

D) Immobilisation of Notch Ligand protein Directly onto a 96-Well Tissue Culture Plate

10 μg of purified hDelta1-IgG4Fc fusion protein was added to sterile PBS in a sterile Eppendorf tube to give a final volume of 1 ml and 100 μl was added to wells of a 96-well tissue culture plate. The lid of the plate was sealed with parafilm and the plate was left at 4° C. overnight or at 37° C. for 2 hours. The protein was then removed and the plate was washed twice with 200 μl of PBS.

Assays were set up in the coated 96-well plates with 2×10⁵ Jurkat cells per well in 100 μl per well of DMEM plus 10%(HI)FCS plus glutamine plus P/S. MW167 was diluted to 20 μM final concentration in complete RPMI from a 10 mM stock solution in DMSO. Control wells were set up with an equivalent dilution of DMSO alone. Plates were left in a CO₂ incubator overnight.

E) Luciferase Assay

Supernatant was removed from all wells leaving 100 μl of cells plus medium behind and 100 μl of SteadyGlo™ luciferase assay reagent (Promega) was added and the cells were left at room temperature for 5 minutes. The mixture was pipetted up and down 2 times to ensure cell lysis and contents from each well were transferred into a white 96-well OptiPlate™ (Packard). Luminescence was measured in a TopCount™ counter (Packard).

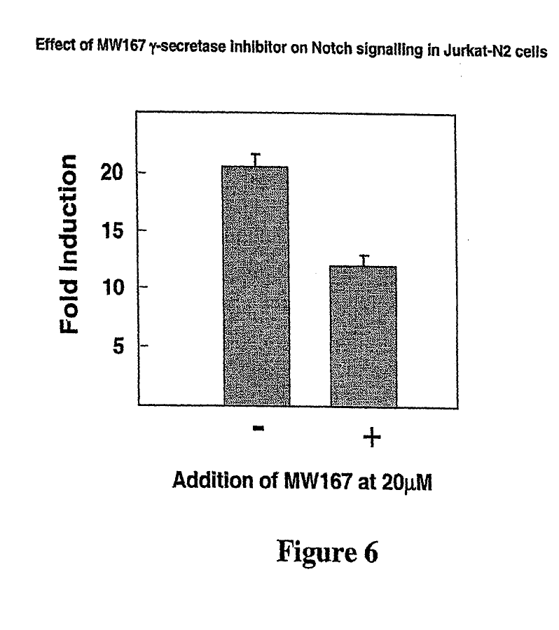

Results of sample assays using the Jurkat cells described above with plate-immobilised hDelta1-IgG4Fc fusion protein, are shown in FIG. 6 (expressed as fold activation of reporter activity compared to cells cultured in the absence of Delta).

The invention is further described by the following numbered paragraphs:

1. Use of a modulator of Notch IC protease activity for the manufacture of a medicament for use in immunotherapy.

2. Use of a modulator of Notch IC protease activity in combination with a modulator of the Notch signalling pathway for the manufacture of a medicament for use in immunotherapy.

3. A use as described in paragraph 1 or paragraph 2 wherein the modulator is an agonist of presenilin or presenilin-dependent gamma-secretase, optionally in combination with an agent capable of up-regulating the Notch signalling pathway.

4. A use as described in paragraph 3 wherein the agonist of presenilin is a polypeptide selected from Nicastrin or ALG-3 or a nucleic acid sequence which encodes therefor.

5. A use as described in paragraph 3 or paragraph 4 wherein the agent capable of up-regulating expression the Notch signalling pathway is a polypeptide selected from Notch ligands, Noggin, Chordin, Follistatin, Xnr3, FGF and derivatives, fragments, variants and homologues thereof, and immunosuppressive cytokines, or a combination thereof, or a nucleic acid sequence which encodes therefor.

6. A use as described in paragraph 1 or paragraph 2 wherein the modulator is an antagonist of presenilin or presenilin-dependent gamma-secretase, optionally in combination with an agent capable of down-regulating the Notch signalling pathway.

7. A use as described in paragraph 6 wherein the antagonist of presenilin is 26S proteasome or Sel 10 or a nucleic acid sequence which encodes therefor.

8. A use as described in paragraph 6 or paragraph 7 wherein the agent capable of down-regulating the Notch signalling pathway is a polypeptide selected from a Toll-like receptor, a cytokine, a bone morphogenetic protein (BMP), a BMP receptor or an activin, or a nucleic acid sequence which encodes therefor.

9. Use of a modulator of Notch IC protease activity for the manufacture of a medicament for modulation of an immune response.

10. Use of a modulator of Notch IC protease activity for the manufacture of a medicament for modulation of an immune response to a selected antigen or antigenic determinant.

11. Use of a modulator of Notch IC protease activity for the manufacture of a medicament for modulation of lymphocyte activity.

12. Use of a modulator of Notch IC protease activity for the manufacture of a medicament for modulation of T-cell activity.

13. Use of a modulator of Notch IC protease activity for the manufacture of a medicament for modulation of effector T-cell activity.

14. Use of a modulator of Notch IC protease activity for the manufacture of a medicament for modulation of helper (Th) T-cell activity.

15. Use of an inhibitor of Notch IC protease activity for the manufacture of a medicament for increase of helper (Th) T-cell activity.

16. Use of an enhancer of Notch IC protease activity for the manufacture of a medicament for decrease of helper (Th) T-cell activity.

17. Use of a modulator of Notch IC protease activity for the manufacture of a medicament for modulation of cytotoxic (Tc) T-cell activity.

18. Use of an inhibitor of Notch IC protease activity for the manufacture of a medicament for increase of cytotoxic (Tc) T-cell activity.

19. Use of an enhancer of Notch IC protease activity for the manufacture of a medicament for decrease of cytotoxic (Tc) T-cell activity.

20. Use of a modulator of Notch IC protease activity for the manufacture of a medicament for modulation of regulatory (T reg) T-cell activity.

21. Use of an inhibitor of Notch IC protease activity for the manufacture of a medicament for inhibition of regulatory (T reg) T-cell activity.

22. Use of an enhancer of Notch IC protease activity for the manufacture of a medicament for enhancement of regulatory (T reg) T-cell activity.

23. Use of a modulator of Notch IC protease activity for the manufacture of a medicament for modulation of Tr1 regulatory T-cell activity.

24. Use of an inhibitor of Notch IC protease activity for the manufacture of a medicament for inhibition of Tr1 regulatory T-cell activity.

25. Use of an enhancer of Notch IC protease activity for the manufacture of a medicament for enhancing Tr1 regulatory T-cell activity.

26. Use of a modulator of Notch IC protease activity for the manufacture of a medicament for modulation of Th3 regulatory T-cell activity.

27. Use of an inhibitor of Notch IC protease activity for the manufacture of a medicament for inhibition of Th3 regulatory T-cell activity.

28. Use of an enhancer of Notch IC protease activity for the manufacture of a medicament for enhancing Th3 regulatory T-cell activity.

29. Use of a modulator of Notch IC protease activity for the manufacture of a medicament for modulation of cytokine expression.

30. Use of a modulator of Notch IC protease activity for the manufacture of a medicament for modulation of lymphokine expression.

31. Use of a modulator of Notch IC protease activity for the manufacture of a medicament for modulation of monokine expression.

32. Use of a modulator of Notch IC protease activity for the manufacture of a medicament for modulation of Notch-mediated cytokine expression.

33. Use of a modulator of Notch IC protease activity for the manufacture of a medicament for modulation of expression of a cytokine selected from IL-10, IL-5, IL-4, IL-2, TNF-alpha, IFN-gamma or IL-13.

34. Use of a modulator of Notch IC protease activity for the manufacture of a medicament for modulation of Notch-mediated expression of a cytokine selected from IL-10, IL-5, IL-4, IL-2, TNF-alpha, IFN-gamma or IL-13.

35. Use of an inhibitor of Notch IC protease activity for the manufacture of a medicament for decrease of IL-10 or IL-4 expression.

36. Use of an inhibitor of Notch IC protease activity for the manufacture of a medicament for decrease of Notch-mediated IL-10 or IL-4 expression.

37. Use of an activator of Notch IC protease activity for the manufacture of a medicament for increase of IL-10 or IL-4 expression.

38. Use of an activator of Notch IC protease activity for the manufacture of a medicament for increase of Notch-mediated IL-10 or IL-4 expression.

39. A use as described in any one of paragraphs 35 to 38 wherein the cytokine is IL-10.

40. A use as described in any one of paragraphs 35 to 38 wherein the cytokine is IL-4.

41. Use of an inhibitor of Notch IC protease activity for the manufacture of a medicament for increase of expression of a cytokine selected from IL-2, IL-5, TNF-alpha, IFN-gamma or IL-13.

42. Use of an inhibitor of Notch IC protease activity for the manufacture of a medicament for increase of Notch-mediated expression of a cytokine selected from IL-2, IL-5, TNF-alpha, IFN-gamma or IL-13.

43. Use of an activator of Notch IC protease activity for the manufacture of a medicament for decrease of expression of a cytokine selected from IL-2, L-5, TNF-alpha, IFN-gamma or IL-13.

44. Use of an activator of Notch IC protease activity for the manufacture of a medicament for decrease of Notch-mediated expression of a cytokine selected from IL-2, IL-5, TNF-alpha, IFN-gamma or IL-13.

45. A use as described in any one of paragraphs 41 to 44 wherein the cytokine is IL-2.

46. A use as described in any one of paragraphs 41 to 44 wherein the cytokine is IL-5.

47. A use as described in any one of paragraphs 41 to 44 wherein the cytokine is TNF-alpha.

48. A use as described in any one of paragraphs 41 to 44 wherein the cytokine is IFN-gamma.

49. A use as described in any one of paragraphs 41 to 44 wherein the cytokine is IL-13.

50. Use of an enhancer of Notch IC protease activity for the manufacture of a medicament for generating an immune modulatory cytokine profile with increased IL-10 expression and reduced IL-5 expression.

51. Use of an enhancer of Notch IC protease activity for the manufacture of a medicament for generating an immune modulatory cytokine profile with increased IL-10 expression and reduced IL-2, IFN-gamma, IL-5, IL-13 and TNF-alpha expression.

52. Use of an inhibitor of Notch IC protease activity for the manufacture of a medicament for generating an immune modulatory cytokine profile with decreased IL-10 expression and increased IL-5 expression.

53. Use of an inhibitor of Notch IC protease activity for the manufacture of a medicament for generating an immune modulatory cytokine profile with decreased IL-10 expression and increased IL-2, IFN-gamma, IL-5, IL-13 and TNF-alpha expression.

54. A use as described in any one of paragraphs 29 to 53 wherein the modulator of Notch IC protease activity modifies cytokine expression in leukocytes, fibroblasts or epithelial cells.

55. A use as described in any one of paragraphs 29 to 53 wherein the modulator of Notch IC protease activity modifies cytokine expression in dendritic cells, lymphocytes or macrophages, or their progenitors or tissue-specific derivatives.

56. A use as described in any one of paragraphs 29 to 53 wherein the modulator of Notch IC protease activity modifies cytokine expression in lymphocytes or macrophages.

57. Use of an enhancer of Notch IC protease activity in the manufacture of a medicament for treatment of inflammation or an inflammatory condition.

58. Use of a combination of: i) an enhancer or inhibitor of Notch IC protease activity; and ii) an antigen or antigenic determinant or a polynucleotide coding for an antigen or antigenic determinant; in the manufacture of a medicament for modulation of the immune system.

59. Use of an enhancer or inhibitor of Notch IC protease activity in the manufacture of a medicament for modulation of the immune system in simultaneous, contemporaneous, separate or sequential combination with an antigen or antigenic determinant or a polynucleotide coding for an antigen or antigenic determinant.

60. A use as described in any one of the preceding paragraphs wherein the modulator of Notch IC protease activity is administered to a patient in vivo.

61. A use as described in any one of paragraphs 1 to 59 wherein the modulator of Notch IC protease activity is administered to a cell ex-vivo.

62. A use as described in any one of the preceding paragraphs wherein the medicament is for use in the treatment of a T cell mediated disease or infection.

63. A use as described in paragraph 62 wherein the T cell mediated disease or infection is any one or more of allergy, autoimmunity, graft rejection, tumour induced aberrations to the T cell and infectious diseases.

64. A use as described in any one of the preceding paragraphs wherein the presenilin modulator is a modulator of Presenilin-1 (PS1) or Presenilin-2 (PS2).

65. A use as described in any one of the preceding paragraphs wherein the presenilin or presenilin-dependent gamma-secretase modulator is selected from polypeptides and fragments thereof, linear peptides, cyclic peptides, and nucleic acids which encode therefor, synthetic and natural compounds including low molecular weight organic or inorganic compounds and antibodies.

66. A method of immunotherapy comprising administering a modulator of Notch IC protease activity.

67. A method of immunotherapy comprising administering a modulator of Notch IC protease activity in combination with a modulator of the Notch signalling pathway.

68. A method as described in paragraph 66 or paragraph 67 wherein the modulator is an agonist of presenilin or presenilin-dependent gamma-secretase, optionally in combination with an agent capable of up-regulating the Notch signalling pathway.

69. A method as described in paragraph 68 wherein the agonist of presenilin is a polypeptide selected from Nicastrin or ALG-3 or a nucleic acid sequence which encodes therefor.

70. A method as described in paragraph 68 or paragraph 69 wherein the agent capable of up-regulating expression the Notch signalling pathway is a polypeptide selected from Notch ligands, Noggin, Chordin, Follistatin, Xnr3, FGF and derivatives, fragments, variants and homologues thereof, and immunosuppressive cytokines, or a combination thereof, or a nucleic acid sequence which encodes therefor.

71. A method as described in paragraph 66 or paragraph 67 wherein the modulator is an antagonist of presenilin or presenilin-dependent gamma-secretase, optionally in combination with an agent capable of down-regulating the Notch signalling pathway.

72. A method as described in paragraph 71 wherein the antagonist of presenilin is 26S proteasome or Sel 10 or a nucleic acid sequence which encodes therefor.

73. A method as described in paragraph 71 or paragraph 72 wherein the agent capable of down-regulating the Notch signalling pathway is a polypeptide selected from a Toll-like receptor, a cytokine, a bone morphogenetic protein (BMP), a BMP receptor or an activin, or a nucleic acid sequence which encodes therefor.

74. A method for modulating an immune response by administering a modulator of Notch IC protease activity.

75. A method for modulating an immune response to a selected antigen or antigenic determinant by administering a modulator of Notch IC protease activity.

76. A method for modulating lymphocyte activity by administering a modulator of Notch IC protease activity.

77. A method for modulating T-cell activity by administering a modulator of Notch IC protease activity.

78. A method for modulating effector T-cell activity by administering a modulator of Notch IC protease activity.

79. A method for modulating helper (Th) T-cell activity by administering a modulator of Notch IC protease activity.

80. A method for increasing helper (Th) T-cell activity by administering an inhibitor of Notch IC protease activity.

81. A method for decreasing helper (Th) T-cell activity by administering an enhancer of Notch IC protease activity.

82. A method for modulating cytotoxic (Tc) T-cell activity by administering a modulator of Notch IC protease activity.

83. A method for increasing cytotoxic (Tc) T-cell activity by administering an inhibitor of Notch IC protease activity.

84. A method for decreasing cytotoxic (Tc) T-cell activity by administering an enhancer of Notch IC protease activity.

85. A method for modulating regulatory (T reg) T-cell activity by administering a modulator of Notch IC protease activity.

86. A method for decreasing regulatory (T reg) T-cell activity by administering an inhibitor of Notch IC protease activity.

87. A method for increasing regulatory (T reg) T-cell activity by administering an enhancer of Notch IC protease activity.

88. A method for modulating Tr1 regulatory T-cell activity by administering a modulator of Notch IC protease activity.

89. A method for inhibiting Tr1 regulatory T-cell activity by administering an inhibitor of Notch IC protease activity.

90. A method for increasing Tr1 regulatory T-cell activity by administering an enhancer of Notch IC protease activity.

91. A method for modulating Th3 regulatory T-cell activity by administering a modulator of Notch IC protease activity.

92. A method for inhibiting Th3 regulatory T-cell activity by administering an inhibitor of Notch IC protease activity.

93. A method for increasing Th3 regulatory T-cell activity by administering an enhancer of Notch IC protease activity.

94. A method for modulating cytokine expression by administering a modulator of Notch IC protease activity.

95. A method for modulating lymphokine expression by administering a modulator of Notch IC protease activity.

96. A method for modulating monokine expression by administering a modulator of Notch IC protease activity.

97. A method for modulating Notch-mediated cytokine expression by administering a modulator of Notch IC protease activity.

98. A method for modulating expression of a cytokine selected from IL-10, IL-5, IL-4, IL-2, TNF-alpha, IFN-gamma or IL-13 by administering a modulator of Notch IC protease activity.

99. A method for modulating Notch-mediated expression of a cytokine selected from IL-1, IL-5, IL-4, IL-2, TNF-alpha, IFN-gamma or IL-13 by administering a modulator of Notch IC protease activity.

100. A method for decreasing IL-10 or IL-4 expression by administering an inhibitor of Notch IC protease activity.

101. A method for decreasing Notch-mediated IL-10 or IL-4 expression by administering an inhibitor of Notch IC protease activity.

102. A method for increasing IL-10 or IL-4 expression by administering an activator of Notch IC protease activity.

103. A method for increasing Notch-mediated IL-10 or IL-4 expression by administering an activator of Notch IC protease activity.

104. A method as described in any one of paragraphs 98 to 103 wherein the cytokine is IL-10.

105. A method as described in any one of paragraphs 98 to 103 wherein the cytokine is IL-4.

106. A method for increasing expression of a cytokine selected from IL-2, IL-5, TNF-alpha, IFN-gamma or IL-13 by administering an inhibitor of Notch IC protease activity.

107. A method for increasing Notch-mediated expression of a cytokine selected from IL-2, IL-5, TNF-alpha, IFN-gamma or IL-13 by administering an inhibitor of Notch IC protease activity.

108. A method for decreasing expression of a cytokine selected from IL-2, IL-5, TNF-alpha, IFN-gamma or IL-13 by administering an activator of Notch IC protease activity. 109. A method for decreasing Notch-mediated expression of a cytokine selected from IL-2, IL-5, TNF-alpha, IFN-gamma or IL-13 by administering an activator of Notch IC protease activity.

110. A method as described in any one of paragraphs 106 to 109 wherein the cytokine is IL-2.

111. A method as described in any one of paragraphs 106 to 109 wherein the cytokine is IL-5.

112. A method as described in any one of paragraphs 106 to 109 wherein the cytokine is TNF-alpha.

113. A use as described in any one of paragraphs 106 to 109 wherein the cytokine is IFN-gamma.

114. A use as described in any one of paragraphs 106 to 109 wherein the cytokine is IL-13.

115. A method for generating an immune modulatory cytokine profile with increased IL-10 expression and reduced IL-5 expression by administering an enhancer of Notch IC protease activity.

116. A method for generating an immune modulatory cytokine profile with increased IL-10 expression and reduced IL-2, IFN-gamma, IL-5, IL-13 and TNF-alpha expression by administering an enhancer of Notch IC protease activity.

117. A method for generating an immune modulatory cytokine profile with decreased IL-10 expression and increased IL-5 expression by administering an inhibitor of Notch IC protease activity.

118. A method for generating an immune modulatory cytokine profile with decreased IL-10 expression and increased IL-2, IFN-gamma, IL-5, IL-13 and TNF-alpha expression by administering an inhibitor of Notch IC protease activity.

119. A method as described in any one of paragraphs 94 to 119 wherein the modulator of Notch IC protease activity modifies cytokine expression in leukocytes, fibroblasts or epithelial cells.

120. A method as described in any one of paragraphs 94 to 119 wherein the modulator of Notch IC protease activity modifies cytokine expression in dendritic cells, lymphocytes or macrophages, or their progenitors or tissue-specific derivatives.

121. A method as described in any one of paragraphs 94 to 119 wherein the modulator of Notch IC protease activity modifies cytokine expression in lymphocytes or macrophages.

122. A method for treating inflammation or an inflammatory condition by administering an enhancer of Notch IC protease activity.

123. A method for modulating the immune system by simultaneously, contemporaneously, separately or sequentially administering a combination of: i) a modulator of Notch IC protease activity; and ii) an antigen or antigenic determinant or a polynucleotide coding for an antigen or antigenic determinant.

124. A method as described in any one of paragraphs 66 to 123 wherein the modulator of Notch IC protease activity is administered to a patient in vivo.

125. A method as described in any one of paragraphs 66 to 123 wherein the modulator of Notch IC protease activity is administered to a cell ex-vivo.

126. A method as described in any one of paragraphs 66 to 125 for the treatment of a T cell mediated disease or infection.

127. A method as described in paragraph 126 wherein the T cell mediated disease or infection is any one or more of allergy, autoimmunity, graft rejection, tumour induced aberrations to the T cell and infectious diseases.

128. A method as described in any one of paragraphs 66 to 127 wherein the presenilin modulator is a modulator of Presenilin-1 (PS1) or Presenilin-2 (PS2).

129. A method as described in any one of paragraphs 66 to 128 wherein the presenilin or presenilin-dependent gamma-secretase modulator is selected from polypeptides and fragments thereof, linear peptides, cyclic peptides, and nucleic acids which encode therefor, synthetic and natural compounds including low molecular weight organic or inorganic compounds and antibodies.

130. A modulator of Notch IC protease activity for use in affecting T cell mediated disease or infection.

131. A modulator of Notch IC protease activity for use in affecting linked suppression.

132. A modulator of Notch IC protease activity for use in affecting infectious tolerance.

133. A modulator of Notch IC protease activity according to any one of paragraphs 130 to 132 in combination with a modulator of the Notch signalling pathway.

134. A method for producing a lymphocyte or antigen presenting cell (APC) having tolerance to an allergen or antigen which method comprises incubating a lymphocyte or APC obtained from a human or animal patient with (i) an agonist of presenilin or presenilin-dependent gamma-secretase and optionally an agent capable of up-regulating endogenous Notch or Notch ligand in the lymphocyte and/or APC and (ii) the allergen or antigen.

135. A method according to paragraph 134 for producing an APC capable of inducing T cell tolerance.

136. A method according to paragraph 134 or paragraph 135 for producing ex vivo a T cell having tolerance to an allergen or antigen which method comprises incubating a T cell obtained from a human or animal patient with an antigen presenting cell (APC) in the presence of (i) an agonist of presenilin or presenilin-dependent gamma-secretase and optionally an agent capable of up-regulating expression of an endogenous Notch or Notch ligand in the APC and/or T cell and (ii) the allergen or antigen.

137. A method for producing a lymphocyte or APC having tolerance to an allergen or antigen which method comprises incubating a lymphocyte or APC obtained from a human or animal patient with a lymphocyte or APC produced by the method of any one of paragraphs 134 to 136.

138. A method according to paragraph 137 for producing ex vivo a T cell having tolerance to an allergen or antigen which method comprises incubating a T cell obtained from a human or animal patient with a T cell produced by the method of any one paragraphs 134 to 136.

139. Use of a lymphocyte or APC produced by the method of any one of paragraphs 134 to 138 in suppressing an immune response in a mammal to the allergen or antigen.

140. A method of treating a patient suffering from a disease characterised by inappropriate lymphocyte activity which method comprises administering to the patient a lymphocyte produced by the method of any one of paragraphs 134 to 138.

141. A method for enhancing the reactivity of a T cell toward a tumour cell which method comprises:

(a) isolating a T cell from a patient having said tumour cell present in their body;

(b) exposing the T cell to a modulator of Notch IC protease activity, optionally in the presence of an agent which is capable of reducing or preventing expression or interaction of an endogenous Notch or Notch ligand in the T cell; and

(c) re-introducing the T cell into the patient;

wherein the T cell comprises a T cell receptor specific for a tumour antigen expressed by the tumour cell.

142. A method for enhancing the reactivity of a T cell toward a tumour cell which method comprises:

(a) isolating an antigen presenting cell (APC) from a tumour present in the body of a patient;

(b) exposing the APC to a modulator of Notch IC protease activity, optionally in the presence of an agent which is capable of reducing or preventing expression or interaction of an endogenous Notch or Notch ligand in the T cell when the T cell is contacted with the APC; and

(c) re-introducing the APC into the patient.

143. A method for enhancing the reactivity of a T cell toward a tumour cell which method comprises:

(a) isolating a tumour cell from a tumour present in the body of a patient;

(b) exposing the tumour cell to a modulator of Notch IC protease activity, optionally in the presence of an agent which is capable of reducing or preventing expression or interaction of an endogenous Notch or Notch ligand in the T cell when the T cell is contacted with the tumour cell; and

(c) re-introducing the tumour cell into the patient.

144. A method according to any one of paragraphs 141 to 143 wherein the T cell is a tumour infiltrating lymphocyte.

145. A method of vaccinating a patient against a tumour which method comprises:

(a) administering a tumour antigen expressed by the tumour to a patient; and

(b) exposing the APC present in the patient to a modulator of presenilin or presenilin-dependent gamma-secretase agent, optionally in the presence of an agent which is capable of reducing or preventing expression, interaction or processing of Notch or a Notch ligand in a T cell.

146. An assay method for modulators of Notch IC protease activity comprising contacting a presenilin or presenilin-dependent gamma-secretase, respectively, in the presence of Notch and a modulator of the Notch signalling pathway, with a candidate compound and determining if the compound affects the Notch signalling pathway.

147. An assay method for identifying substances that affect the interaction of a presenilin interacting protein or presenilin-dependent gamma-secretase interacting protein with a presenilin protein or presenilin-dependent gamma-secretase, respectively, comprising:

(a) providing a preparation containing: a presenilin protein or presenilin-dependent gamma-secretase; a presenilin-interacting protein or presenilin-dependent gamma-secretase, respectively; and a candidate substance; and

(b) detecting whether said candidate substance affects said interaction of said presenilin-interacting protein or presenilin-dependent gamma-secretase-interacting protein with said presenilin protein or presenilin-dependent gamma-secretase.

148. An assay method according to paragraph 147 wherein the presenilin-interacting protein is Notch or a member of the Notch signalling pathway.

149. An assay method according to any one of paragraphs 146 to 148 wherein the assay is conducted using an immune cell.

150. Use of a presenilin or presenilin-dependent gamma-secretase modulator identifiable using the assay method of any of paragraphs 146 to 149 in the use or method of any one of paragraphs 1 to 145.

151. A kit comprising in one or more containers (a) a modulator of the Notch signalling pathway and (b) a modulator of presenilin or presenilin-dependent gamma-secretase activity.

152. A product comprising: i) a modulator of Notch IC protease activity; and ii) an antigen or antigenic determinant or a polynucleotide coding for an antigen or antigenic determinant; as a combined preparation for simultaneous, contemporaneous, separate or sequential use for modulation of the immune system.

153. A pharmaceutical composition comprising: i) a modulator of Notch IC protease activity; and ii) an antigen or antigenic determinant or a polynucleotide coding for an antigen or antigenic determinant; as a combined preparation for simultaneous, contemporaneous, separate or sequential use for modulation of the immune system.

154. A pharmaceutical composition comprising: i) a modulator of Notch IC protease activity; ii) an antigen or antigenic determinant or a polynucleotide coding for an antigen or antigenic determinant; and iii) a pharmaceutically acceptable carrier.

155. A product as described in any one of paragraphs 152 to 154 for increasing effector T cell activity.

156. A pharmaceutical kit comprising a modulator of Notch IC protease activity and an antigen or antigenic determinant or a polynucleotide coding for an antigen or antigenic determinant.

157. The use of a modulator of Notch IC protease activity in the manufacture of a medicament for use as an immunostimulant.

158. The use of a modulator of Notch IC protease activity in the manufacture of a medicament for use in vaccination against a pathogen.

159. The use of a modulator of Notch IC protease activity in the manufacture of a medicament for use in vaccination against a tumour or pathogen.

160. The use of a modulator of Notch IC protease activity in the manufacture of a medicament for increasing the immune response against a tumour or pathogen antigen or antigenic determinant.

161. A method for stimulating the immune system by administering a modulator of Notch IC protease activity

162. A method for vaccinating a subject against a tumour or pathogen by administering a modulator of Notch IC protease activity

163. A method for increasing an immune response of a subject against a tumour or pathogen by administering a modulator of Notch IC protease activity

164. A method for increasing the immune response of a subject to a tumour or pathogen antigen or antigenic determinant comprising administering an effective amount of a modulator of Notch IC protease activity simultaneously, contemporaneously, separately or sequentially with said tumour or pathogen antigen or antigenic determinant or simultaneously, contemporaneously, separately or sequentially with a polynucleotide coding for said tumour or pathogen antigen or antigenic determinant.

165. An adjuvant composition comprising a modulator of Notch IC protease activity.

166. A vaccine composition comprising an adjuvant composition as described in paragraph 165 and a tumour or pathogen antigen or antigenic determinant or a polynucleotide coding for a tumour or pathogen antigen or antigenic determinant.

167. A vaccine composition as described in paragraph 166 comprising a pathogen antigen or antigenic determinant in the form of a viral, fungal, parasitic or bacterial antigen or antigenic determinant or a polynucleotide coding for a viral, fungal, parasitic or bacterial antigen or antigenic determinant.

168. A product comprising: i) a modulator of Notch IC protease activity; and ii) a tumour or pathogen antigen or antigenic determinant or a polynucleotide coding for a tumour or pathogen antigen or antigenic determinant; as a combined preparation for simultaneous, contemporaneous, separate or sequential use for modulation of the immune system.

169. A product as described in paragraph 168 for increasing effector T cell activity.

### REFERENCES INCORPORATED HEREIN BY REFERENCE THERETO

Hadland et al. (2001) Proc Natl Acad Sci 98(13):7487-91.

DeStrooper et al. (1998) Nature 391:387-390.

Selkoe (2000) Curr. Opin. Neurobiol. 10:50-57.

Citron et al. (1996) Proc Natl Acad Sci 93(23):13170-5.

Wolfe et al. (1998) J Med Chem 41(1):6-9.

Sinha and Liederburg (1999) Proc Natl Acad Sci 96:11049.

Esler et al. (2000) Nature Cell Biology 2:428-434.

Figueiredo-Pereira et al. (1999) J Neurochem 72:1417.

Higaki et al (1999) J Med Chem 42:3889.

Murphy et al. (2000) J Biol Chem 275:26277.

Rishton et al. (2000) J Med Chem 43:2297.

Ellisen et al. (1991) Cell 66:649.

Qin et al. (1993) Science 253:974.

Tamura et al. (1995) Curr. Biol. 5:1416-1423.

Artavanis-Tsakomas et al. (1995) Science 268:225-232.

Artavanis-Tsakomas et al. (1999) Science 284:770-776.

Lieber et al. (1993) Genes Dev 7(10):1949-65.

Schroeter et al. (1998) Nature 393(6683):382-6.

Struhl et al. (1998) Cell 93(4):649-60.

Weinmaster (2000) Curr. Opin. Genet. Dev. 10:363-369.

Lu et al. (1996) Proc Natl Acad Sci 93(11):5663-7.

Munro and Freeman (2000) Curr. Biol. 10:813-820.

Ju et al. (2000) Nature 405:191-195.

Moloney et al. (2000) Nature 406:369-375.

Brucker et al. (2000) Nature 406:411-415.

Panin et al. (1997) Nature 387:908-912.

Hicks et al. (2000) Nat. Cell. Biol. 2:515-520.

Irvine (1999) Curr. Opin. Genet. Devel. 9:434-441.

Devereux et al. (1984) Nucleic Acid Research 12:87.

Atschul et al. (1990) J. Mol. Biol. 403-410.

Inaba et al. (1992) J. Exp. Med. 175:1157-1167.

Caux et al. (1992) Nature 360:258-261.

Coffin et al. (1998) Gene Therapy 5:718-722.

Chee et al. (1996) Science 274:601-614.

Camilli et al. (1994) Proc Natl Acad Sci USA 91:2634-2638.

Hoyne et al. (2000) Immunology 100:281-288.

Hoyne et al. (2001)—reference in press.

D'Adamino et al. (1997) Semin Immunol 9(1):17-23.

Lacana et al. (1997) 158(11):5129-35.

Fagan et al. (2001) 26(4):213-4.

Gang et al. (2000) Nature 407:48-54.

Passer et al. (1999) J Biol Chem 274(34):24007-13.

Choi et al. (2001) J Biol Chem 276(22):19197-204.

Kesavapany et al. (2001) Eur J Neurosci 13(2):241-7.

Fraser et al. (1998) Neurobiol Aging 19(1 Suppl):S19-21.

Capell et al. (2000) Nat Cell Biol 2:205-11.

Sallusto & Lanzavecchia (1994) J. Exp. Med. 179:1109-1118.

Vose et al. (1977) Eur. J. Immunol. 7:353-357.

Belldegrun et al. (1988) Cancer Res. 48: 206-214.

Belldegrun et al. (1989) J. Immunol. 134: 4520-4526.

Spiess et al. (1987) J. Nat. Cancer Inst. 79: 1067-1075.

Dunbar et al. (1998) Curr. Biol. 8: 413-416.

Romero et al. (1998) J. Exp. Med. 188: 1641-1650.

Phizicky & Fields (1995) Microbio. Rev. 59(1):94-123.

Fields & Stemglanz (1994) Trends Genet. 10(8):286-292.

Altman et al. (1996) Science 274:94-96.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

We claim:

1. A method for modulating an immune response comprising administering a modulator of Notch intracellular domain (Notch IC) protease activity.

2. The method of claim 1, wherein the modulator of Notch IC is an agonist of presenilin or presenilin-dependent gamma-secretase.

3. The method of claim 2, wherein the presenilin is Presenilin-1 (PS1) or Presenilin-2 (PS2).

4. The method of claim 2, wherein the agonist is selected from the group consisting of polypeptides, fragments thereof, linear peptides, cyclic peptides, nucleic acids encoding therefor, synthetic compounds, natural compounds, low molecular weight organic compounds, low molecular weight inorganic compounds and antibodies.

5. The method of claim 1, further comprising administering a modulator of Notch signalling pathway.

6. The method of claim 5, wherein the modulator of Notch IC is an agonist of presenilin or presenilin-dependent gamma-secretase.

7. The method of claim 6, wherein the presenilin is Presenilin-1 (PS1) or Presenilin-2 (PS2).

8. The method of claim 6, wherein the agonist is selected from the group consisting of polypeptides, fragments thereof, linear peptides, cyclic peptides, nucleic acids encoding therefor, synthetic compounds, natural compounds, low molecular weight organic compounds, low molecular weight inorganic compounds and antibodies.

9. The method of claim 6, wherein the modulator of the Notch signalling pathway is an agent that up-regulates the Notch signalling pathway.

10. The method of claim 7, wherein the agonist of presenilin is Nicastrin or ALG-3, or a nucleic acid sequence encoding therefor.

11. The method of claim 6, wherein the modulator of the Notch signalling pathway is an agent that down-regulates the Notch signalling pathway.

12. The method of claim 11, wherein the agonist of presenilin is 26S proteasome or Sel 10 or a nucleic acid sequence encoding therefor.

13. The method of claim 7, wherein the agent that up-regulates the Notch signalling pathway is a polypeptide selected from the group consisting of Notch ligands, Noggin, Chordin, Follistatin, Xnr3, FGF, derivatives, fragments, variants and homologues thereof, and immunosuppressive cytokines, or is a combination thereof, or is a nucleic acid sequence encoding therefor.

14. The method of claim 11, wherein the agent that down-regulates the Notch signalling pathway is a polypeptide selected from the group consisting of a Toll-like receptor, a cytokine, a bone morphogenetic protein (BMP), a BMP receptor and an activin, or is a nucleic acid sequence encoding therefor.

15. The method of claim 1, wherein the immune response is to a selected antigen or antigenic determinant.

16. The method of claim 15, wherein the selected antigen or antigenic determinant, or a nucleic acid encoding the antigen or antigenic determinant, is administered simultaneously, contemporaneously, separately or sequentially with the modulator.

17. The method of claim 15, wherein the antigen or antigenic determinant is a tumour antigen or antigenic determinant or an antigen or antigenic determinant of a pathogen.

18. The method of claim 1, wherein modulating the immune response comprises modulating lymphocyte activity.

19. The method of claim 1, wherein modulating the immune response comprises modulating T-cell activity.

20. The method of claim 19, wherein the T-cell is an effector T-cell.

21. The method of claim 19, wherein the T-cell is a helper (Th) T-cell.

22. The method of claim 21, wherein the modulator is an inhibitor of Notch IC protease activity, and wherein helper (Th) T-cell activity is increased.

23. The method of claim 21, wherein the modulator is an enhancer of Notch IC protease activity, and wherein helper (Th) T-cell activity is decreased.

24. The method of claim 19, wherein the T-cell is a cytotoxic (Tc) T-cell.

25. The method of claim 24, wherein the modulator is an inhibitor of Notch IC protease activity, and wherein cytotoxic (Tc) T-cell activity is increased.

26. The method of claim 24, wherein the modulator is an enhancer of Notch IC protease activity, and wherein cytotoxic (Tc) T-cell activity is decreased.

27. The method of claim 19, wherein the T-cell is a regulatory (T reg) T-cell.

28. The method of claim 27, wherein the modulator is an inhibitor of Notch IC protease activity, and wherein regulatory (T reg) T-cell activity is decreased.

29. The method of claim 27, wherein the modulator is an enhancer of Notch IC protease activity, and wherein regulatory (T reg) T-cell activity is increased.

30. The method of claim 19, wherein the T-cell is a Tr1 regulatory T-cell.

31. The method of claim 30, wherein the modulator is an inhibitor of Notch IC protease activity, and wherein Tr1 regulatory T-cell activity is decreased.

32. The method of claim 30, wherein the modulator is an enhancer of Notch IC protease activity, and wherein Tr1 regulatory T-cell activity is increased.

33. The method of claim 19, wherein T-cell is a Th3 regulatory T-cell.

34. The method of claim 33, wherein the modulator is an inhibitor of Notch IC protease activity, and wherein Th3 regulatory T-cell activity is decreased.

35. The method of claim 33, wherein the modulator is an enhancer of Notch IC protease activity, and wherein Th3 regulatory T-cell activity is increased.

36. The method of claim 1, wherein the modulator is administered to a subject in vivo.

37. The method of claim 1, wherein the modulator is administered to a cell ex vivo.

38. The method of claim 1, wherein modulating the immune response treats a T cell mediated disease or infection.

39. The method of claim 38, wherein the T cell mediated disease or infection is one or more of allergy, autoimmunity, graft rejection, tumour induced aberrations to the T cell, and infectious diseases.

40. The method of claim 1, wherein the modulator is an enhancer of Notch IC protease activity, when wherein modulating the immune response treats inflammation or an inflammatory condition.

41. The method of claim 1, wherein a subject's immune system is stimulated.

42. A method for modulating cytokine expression comprising administering a modulator of Notch intracellular domain (Notch IC) protease activity.

43. The method of claim 42, wherein the cytokine is a lymphokine.

44. The method of claim 42, wherein the cytokine is a monokine.

45. The method of claim 42, wherein the cytokine expression is Notch-mediated cytokine expression.

46. The method of claim 42, wherein the cytokine is selected from the group consisting of IL-10, IL-5, IL-4, IL-2, TNF-alpha, IFN-gamma and IL-13.

47. The method of claim 46, wherein the cytokine is IL-10 or IL-4, wherein the modulator is an inhibitor of Notch IC protease activity, and wherein expression of the cytokine is decreased.

48. The method of claim 46, wherein the cytokine is IL-10 or IL-4, wherein the modulator is an enhancer of Notch IC protease activity, and wherein expression of the cytokine is increased.

49. The method of claim 46, wherein the cytokine is IL-10.

50. The method of claim 46, wherein the cytokine is IL-4.

51. The method of claim 46, wherein the cytokine is IL-2, IL-5, TNF-alpha, IFN-gamma or IL-13, wherein the modulator is an inhibitor of Notch IC protease activity, and wherein expression of the cytokine is increased.

52. The method of claim 46, wherein the cytokine is IL-2, IL-5, TNF-alpha, IFN-gamma or IL-13, wherein the modulator is an enhancer of Notch IC protease activity, and wherein expression of the cytokine is decreased.

53. The method of claim 46, wherein the cytokine is IL-2.

54. The method of claim 46, wherein the cytokine is IL-5.

55. The method of claim 46, wherein the cytokine is TNF-alpha.

56. The method of claim 46, wherein the cytokine is IFN-gamma.

57. The method of claim 46, wherein the cytokine is IL-13.

58. The method of claim 46, wherein the modulator is an enhancer of Notch IC protease activity, and wherein IL-10 expression is increased and IL-5 expression is decreased.

59. The method of claim 46, wherein the modulator is an enhancer of Notch IC protease activity, and wherein IL-10 expression is increased and IL-2, IFN-gamma, IL-5, IL-13 and TNF-alpha expression are decreased.

60. The method of claim 46, wherein the modulator is an inhibitor of Notch IC protease activity, and wherein IL-10 expression is decreased and IL-5 expression is increased.

61. The method of claim 46, wherein the modulator is an inhibitor of Notch IC protease activity, and wherein IL-10 expression is decreased and IL-2, IFN-gamma, IL-5, IL-13 and TNF-alpha expression are increased.

62. The method of claim 42, wherein cytokine expression is modified in leukocytes, fibroblasts or epithelial cells.

63. The method of claim 42, wherein cytokine expression is modified in cells selected from the group consisting of dendritic cells, lymphocytes, macrophages, progenitors therof, and tissue-specific derivatives thereof.

64. The method of claim 63, wherein the cells are lymphocytes or macrophages.

65. The method of claim 42, wherein the modulator is administered to a subject in vivo.

66. The method of claim 42, wherein the modulator is administered to a cell ex vivo.

67. The method of claim 42, wherein modulating the immune response treats a T cell mediated disease or infection.

68. The method of claim 67, wherein the T cell mediated disease or infection is one or more of allergy, autoimmunity, graft rejection, tumour induced aberrations to the T cell, and infectious diseases.

69. The method of claim 42, wherein the modulator is an enhancer of Notch IC protease activity, when wherein modulating the immune response treats inflammation or an inflammatory condition.

70. A modulator of Notch IC protease activity for use in affecting (i) T cell mediated disease or infection, (ii) linked suppression or (iii) infectious tolerance.

71. A composition comprising the modulator of claim 70 and a modulator of the Notch signalling pathway.

72. A method for producing a lymphocyte or antigen presenting cell (APC) having tolerance to an allergen or antigen, which method comprises incubating a lymphocyte or APC obtained from a human or animal subject with (i) an agonist of presenilin or presenilin-dependent gamma-secretase and, optionally, an agent that up-regulates endogenous Notch or Notch ligand in the lymphocyte or APC and (ii) the allergen or antigen, thereby producing a lymphocyte or APC having tolerance to the allergen or antigen.

73. The method of claim 72, wherein an APC capable of inducing T cell tolerance is produced.

74. A method for producing a lymphocyte or APC having tolerance to an allergen or antigen, which method comprises incubating a lymphocyte or APC obtained from a human or animal subject with the lymphocyte or APC produced by the method of claim 72.

75. A method of suppressing an immune response to an allergen or antigen in a mammal, which method comprises administering to the mammal a lymphocyte or APC produced by the method of claim 72.

76. A method of treating a subject having a disease characterised by inappropriate lymphocyte activity, which method comprises administering to the subject a lymphocyte produced by the method of claim 72.

77. A method of treating a subject having a disease characterised by inappropriate lymphocyte activity, which method comprises administering to the subject a lymphocyte produced by the method of claim 74.

78. A method for producing, ex vivo, a T cell having tolerance to an allergen or antigen, which method comprises incubating a T cell obtained from a human or animal subject with an antigen presenting cell (APC), in the presence of (i) an agonist of presenilin or presenilin-dependent gamma-secretase and, optionally, an agent that up-regulates expression of an endogenous Notch or Notch ligand in the APC or T cell and (ii) the allergen or antigen, thereby producing a T cell having tolerance to the allergen or antigen.

79. A method for producing a lymphocyte or APC having tolerance to an allergen or antigen, which method comprises incubating a lymphocyte or APC obtained from a human or animal subject with the T cell produced by the method of claim 77.

80. A method of treating a subject having a disease characterised by inappropriate lymphocyte activity, which method comprises administering to the subject a lymphocyte produced by the method of claim 79.

81. A method for enhancing the reactivity of a T cell toward a tumour cell which method comprises:
a. isolating a T cell, antigen presenting cell (APC) or tumour cell from a subject having a tumour cell present in their body;
b. exposing the T cell, APC or tumour cell to a modulator of Notch IC protease activity, optionally in the presence of an agent which reduces or prevents expression of or interaction between an endogenous Notch or Notch ligand in a T cell; and
c. re-introducing the T cell, APC or tumour cell into the subject.

82. The method of claim 81, wherein the T cell is a tumour infiltrating lymphocyte.

83. A method of vaccinating a subject against a tumour, which method comprises:
a. administering a tumour antigen expressed by cells of the tumour to the subject; and
b. exposing an APC present in the subject to a modulator of presenilin or presenilin-dependent gamma-secretase agent, optionally in the presence of an agent which reduces or prevents expression of, interaction between or processing of Notch and/or a Notch ligand in a T cell.

84. An assay for identifying modulators of Notch IC protease activity, wherein the assay comprises contacting a presenilin or presenilin-dependent gamma-secretase, in the presence of Notch and a modulator of the Notch signalling pathway, with a candidate compound and determining whether the candidate compound affects the Notch signalling pathway, thereby identifying a modulator of Notch IC protease activity, if Notch signalling is different when the candidate compound is present than when it is absent.

85. The assay of claim 84, wherein the assay is conducted using an immune cell.

86. An assay for identifying a substance that affects the interaction of a presenilin interacting protein or presenilin-dependent gamma-secretase interacting protein with a presenilin protein or presenilin-dependent gamma-secretase, respectively, comprising:
a. providing a preparation containing: a presenilin protein or presenilin-dependent gamma-secretase; a presenilin-interacting protein or presenilin-dependent gamma-secretase, respectively; and a candidate substance; and
b. detecting whether the candidate substance affects the interaction of presenilin-interacting protein or presenilin-dependent gamma-secretase-interacting protein with presenilin protein or presenilin-dependent gamma-secretase;
thereby identifying a candidate substance that affects the interaction of a presenilin interacting protein or presenilin-dependent gamma-secretase interacting protein with a presenilin protein or presenilin-dependent gamma-secretase, respectively, if the interaction is different when the candidate substance is present in the preparation than when it is absent in the preparation.

87. The assay of claim 86, wherein the assay is conducted using an immune cell.

88. The assay of claim 86, wherein the presenilin-interacting protein is Notch or a member of the Notch signalling pathway.

89. A composition for immunomodulation comprising: (i) a modulator of Notch IC protease activity, and (ii) an antigen or antigenic determinant or a polynucleotide encoding an antigen or antigenic determinant, wherein (i) and (ii) are administered simultaneously, contemporaneously, separately or sequentially.

90. The composition of claim 89, further comprising a pharmaceutically acceptable carrier.

91. The composition of claim 89, wherein immunomodulation comprises increasing effector T cell activity.

92. The composition of claim 89, wherein the antigen or antigenic determinant is a tumour antigen or antigenic determinant or an antigen or antigenic determinant of a pathogen.

93. A kit comprising, in one or more containers, (i) a modulator of Notch signalling pathway and (ii) a modulator of presenilin or presenilin-dependent gamma-secretase activity.

94. An adjuvant composition comprising a modulator of Notch IC protease activity.

95. A vaccine composition comprising the adjuvant composition of claim 94 and a tumour or pathogen antigen or antigenic determinant or a polynucleotide encoding a tumour or pathogen antigen or antigenic determinant.

96. The vaccine composition of claim 95 comprising a pathogen antigen or antigenic determinant in the form of a viral, parasitic or bacterial antigen or antigenic determinant, or a polynucleotide encoding a viral, fungal, parasitic or bacterial antigen or antigenic determinant.


SEQUENCE LISTING (9 sequences)

SEQ ID NO: 1 - DNA, 24 bp, Artificial sequence
  Oligonucleotide
tcgtcgttttgtcgttttgtcgtt

SEQ ID NO: 2 - protein, 864 aa, Artificial sequence
  hDelta1-IgG4Fc fusion protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHGYVCECARGYGGPNCQFLLPELPPGPAVVDLTEKLEASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - DNA, 26 bp, Artificial sequence
  Adenovirus major late promoter TATA-box motif with BglII and Hind III cohesive ends
gatctggggggctataaaagggggta

SEQ ID NO: 4 - DNA, 26 bp, Artificial sequence
  Adenovirus major late promoter TATA-box motif with BglII and Hind III cohesive ends
agcttacccccttttatagcccccca

SEQ ID NO: 5 - DNA, 50 bp, Artificial sequence
  TP1 promoter sequence with BamH1 and BglII cohesive ends
gatcccgactcgtgggaaaatgggcggaagggcaccgtgggaaaatagta

SEQ ID NO: 6 - DNA, 50 bp, Artificial sequence
  TP1 promoter sequence with BamH1 and BglII cohesive ends
gatctactattttcccacggtgcccttccgcccattttcccacgagtcgg

SEQ ID NO: 7 - protein, 43 aa, Artificial sequence
  DSL domain
CXXXXXXXXCXXXCXXXXXXXXXXXCXXXXXXXCXXXXXXXXC

SEQ ID NO: 8 - protein, 43 aa, Artificial sequence
  DSL domain
CXXXYYXXXCXXXCRPRBDXFGHXXCXXXGXXXCXXGWXGXXC

SEQ ID NO: 9 - protein, 175 aa, Artificial sequence
  Typical EGF-like domain
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX